SEQUENCE LISTING (11 sequences)

SEQ ID NO: 1 - protein, 599 aa, Gallus gallus
MVKHQPLQYYEPQLCLSCLTGIYGCRWKRYQRSHDDTTKWERLWFLILTSSFFLTLVWFYFWWEVHNDYNEINWFLYNRMGYWSDWSIPILVTTAAGFTYITVLLILALCHIAVGQQMNLHWLHKIGLMTTLITTVVTMSSIAQLWDDEWEMVFISLQATAPFLHIGALAAVTALSWLIAGQFARMEKATSQMLMVTAYLAVVVALYLVPLTISSPCIMEKKALGPKPAIIGHRGAPMLAPENTLMSFQKAVEQKIYGVQADVILSYDGVPFLMHDKTLRRTTNVEEVFPGRAYEHSSMFNWTDLEMLNAGEWFLRNDPFWTAGSLSRSDYLEAANQSVCKLADMLEVIKDNTSLILNFQDLPPDHPYYTSYINITLKTILASGIQQQAVMWLPDTERQLVRQIAPAFQQTSGLKLDAERLREKGIVKLNLRYTKVTNEDVRDYMAANLSVNLYTVNEPWLYSILWCTGVPSVTSDSSHVLRKVPFPIWLMPPDEYRLIWITSDLISFIIIVGVFIFQNYHNDQWRLGSIRTYNPEQIMLSAAVRRSSRDVKIMKEKLIFSEINNGVETTDELSLCSENGYANEMVTPTDHRDTRLRMN

SEQ ID NO: 2 - DNA, 4525 bp, Gallus gallus
  modified_base (2049): a, c, g, t, unknown or other
catctctgggaggggacgggggtcccccccgcatcggagctgggcgacctggaggatcacccggcggcagcttgcagggctctcccaggacctgatggctttctgatacgtcgctcctgaggggctggcaggagcatggttaaacaccaaccgctgcagtactacgagccgcagctatgcttgtcgtgcctgaccgggatctacggctgccggtggaaacggtaccagcggtcacatgatgacactaccaagtgggagcgcctttggttcctcatcctcacctcatccttcttcctgactctggtctggttttacttctggtgggaagttcacaatgactacaatgaaatcaactggtttttatataaccgaatgggctattggagtgactggtccattccaatacttgtaacaactgctgctggctttacttacattacagtgttattgatactagcactatgtcacatagctgtgggacagcaaatgaacctacactggcttcacaagattggcctgatgacgaccttgataaccaccgtggtgacaatgtcatcgatagcacagctctgggacgatgaatgggagatggtatttatttcactgcaggccacagcccctttcctgcacataggagctcttgcagcagtcacagccttgtcgtggttgatagcggggcagtttgcacgaatggagaaagccacttctcagatgctgatggtcactgcatacctcgcagtcgtagttgcactttacctcgtccccctcaccatctcatccccttgcataatggagaagaaggctctgggaccaaagccagccatcataggtcaccgtggggcgccgatgttggcaccagaaaataccctgatgtccttccagaaggcggtggagcagaagatatatggagtccaggctgatgtcatattaagctatgatggagtgccatttctgatgcatgacaagacactcaggagaacaacgaacgtggaggaagtgtttccagggcgggcctacgaacactcttccatgttcaactggactgacctggaaatgctcaatgctggagagtggttcctacggaacgaccctttctggacagctggatccctctctaggtctgactatttggaagctgcaaaccagtctgtctgcaagctagcagatatgctggaggtgatcaaggataacacatcactcatcctgaacttccaggacctgccaccagatcatccttattacacttcttacatcaacatcaccctgaagaccatcctggcgtcgggaattcagcaacaagctgtgatgtggctgccggacacggagcggcagctggtcaggcagattgctccagctttccagcagacttctggcctcaagttggatgcagagcgcttgagagagaaaggcattgtgaagctcaacctgcgctacaccaaggtcacgaatgaggatgtcagagactacatggcagcaaacctgagcgtgaatctctacactgtgaatgagccctggctgtactccatcctgtggtgcactggtgtcccgtcggtcacctccgacagctcccacgtccttcgcaaggtgccttttcccatctggttgatgcctccagacgagtaccgcctaatctggatcacatctgatctcatttcatttatcataattgtaggagtttttatattccagaactatcacaatgatcagtggcgcttaggaagcatcaggacctacaacccagagcagatcatgctcagcgctgccgtccgccggtccagtcgggatgtcaagatcatgaaggagaagctgatcttctcggagatcaacaacggcgtggagaccacagacgagctgtccctgtgctctgaaaatggctacgccaacgagatggtcacccccacggatcatcgggacaccaggctgcggatgaactgaggggctccagcccaaccccacgcgtggccaaccccacatgcgctctggcaacgagagctgcgctctcctgcttgccatgggatgggaaacttggaggaactgctggccgggcanaggtcccttcccattccaccatcagtggctgctccacctctctggttctgagattgaggggcttttttttttgtaaatatgtagaaaatctgaatttttttttttgtcttttttttttttttttttcctcgagtgtaattttttccagggcatgagtggaaataccgtgttcatcaaaccctctacgttatggctgaagttatttgaaccttgacgtgacagcctgagtccccctggggggagtttgggacagttccccccaccctgctcccttccttcccttgatgcatcgaccgtgtagctgaattgtttaaactgtatgttgtgaagttctcgtggagtgtcgtttgtttttttttttctgttttatttttgttcccccctcccccctccacctttcccttcctgggcaatgaagtgttagctgtaattgaacaggaggctgcttggaggagagaagctttccttggacaggagatggctctgctcttccgtgttgcagttcagagctggaggagtggcactggctcanacacttgggcacgttccctctgtcccattccccatccccaaaacttggatgtcgacagaaaaatggccttcttgaaatgtctgtaggcgaatcctgggcggtagctgcagattggcctctgctggcagcagtgctttgattttgttgttctttttctatcttctccatgagttgggctccggtgggctcctggatggcttgcaaaaagcaatgccacgggcactgtcactgctcccatctggctgaagtgcctgcacttgcagttgctccttacttcgctgcatccatgcaggaggaggaggagcagcagtgtgagcccctctgtgcccaacagatgcccctgggtgaaggagatgtaaatagtttggaagctgaagtagcccaggacagtaatttattcctgcaaggaggtgtttcctacttgctttaaaaccagcatctttaaagctgcatgttggcatcctccaaaggagcagcggtcttgcagcagatgcacacacccagtgcaaaaggcttgaaaccaaactcgtagagcagaggtttagagaagcatgaaataaagcttagaagccacactgatggtggttgtgttttgtccccacactgcatacagcccagggctgggatgctcagctccctgctctcagtctccaaggtctcgccgggcanagcctgtttttgggtcaccgtgctctttgtgtggatctggttcccaaggcagctgaatccagctggaaggctgacagtgacatgaatggctcttggctcggttcctgtggccttccggtgttgggtcactccttccctcaccggatctcggttggtacaacgtgcaaagtgggaaatcccattgagcacgagcttcctttgaagcttggtgtgttccaccttggaaatgccaagaccataggagttgtttcccgtggaggaaatgtgacccaaagctcaggctttgcgctgagcctgcttgctgagccctgggagaagtcgccttgcagaaaagaagggaggtttgggggatgctgatgtgcctcctggtggactcctaaagcaaacctgcaaaagccagaagcaagccacagcaatcagcctcgagcaggtcgtccttgccgtggcagcactgaagttttgaaccttgaatgtactgaagtagcgtattgaaacctcagaatcttgggttcggtgcatgagaattagagtgcagaagtggtcgttgcagcttgtgcaaaggaaaagaaggcatgccaaagtgacataaggaacatacctctgttaataacgcaagtaagcaaatctgaattgtgatgctttagtacgcaccttctaagcagcacaaattgagacttcaatttgtctgcatctaccttgggttttcatgaagtagatggaacgaggcctagtccacgctccctttatcctcctcaaatctgatctttgtatgcaacttcttgagtctgtgaaacgtggtggggggttacttttttttttcctgcagaatcacaaggttttggggatttgaaggcttttgagataaaagttgcttttcccttaatgaggcgccgcactgaagcgttcctggcggattgtcattgccagtgagagctgttgttgaatcctggcatctgttgataatccctgcatgttctgttattatcctcggcgatgacgttattgaaaggtgaagtgcttaaaagcttggaagtgttaaaagcaatccaaccaagagccctgtttcacaagtcattttttttatgatgagacccaaacttatggtaatgcccgactgttaccgttatcgtcctgaagatttgctacttttttttttttttaagggaatatacttttttgatgaaaatagagctagaccaaaatgaactctgcgatgctctgtgaaataaactgtgcagtattcccctgccaaaaaaaaaaaaaaaaagaaaaaaa

SEQ ID NO: 3 - RNA, 21 nt, Artificial Sequence
  Description of Artificial Sequence Synthetic oligonucleotide
aauccagcuggaaggcugaca

SEQ ID NO: 4 - RNA, 21 nt, Artificial Sequence
  Description of Artificial Sequence Synthetic oligonucleotide
aaagcucaggcuuugcgcuga

SEQ ID NO: 5 - protein, 27 aa, Homo sapiens
GCRCVELDCWKGRTAEEEPVITHGFTM

SEQ ID NO: 6 - protein, 25 aa, Homo sapiens
GCRCIELDCWDGPDGMPVIYHGHTL

SEQ ID NO: 7 - protein, 25 aa, Homo sapiens
GCRCLELDCWDGPNQEPIIYHGYTF

SEQ ID NO: 8 - protein, 25 aa, Homo sapiens
GCRSVELDCWDGDDGMPIIYHGHTL

SEQ ID NO: 9 - protein, 25 aa, Artificial Sequence
  Description of Artificial Sequence Synthetic consensus peptide
GCRCVELDCWDGPDGMPVIYHGHTL

SEQ ID NO: 10 - protein, 25 aa, Rattus sp.
GATGVELDIEFTSDGVPVLMHDNTV

SEQ ID NO: 11 - protein, 25 aa, Gallus gallus
KIYGVQADVILSYDGVPFLMHDKTL

(19) United States
(12) Patent Application Publication
(10) Pub. No.: US 2010/0319073 A1
(43) Pub. Date: Dec. 16, 2010

(54) GDE Compositions and Methods

(75) Inventors: Shanthini Sockanathan, Baltimore, MD (US); Meenakshi Rao, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/992,877

(22) PCT Filed: Sep. 29, 2006
(86) PCT No.: PCT/US06/38324

§ 371 Date: Aug. 5, 2009

(30) Foreign Application Priority Data
Sep. 29, 2005 (US) 60721780

(51) Int. Cl. G01N 33/00; C12N 5/07; C12N 5/077; C12N 5/079; A01K 67/033; C12N 5/0797; C12N 15/63; C12N 5/10; G01N 33/566; A61K 39/395; A61K 31/7088; A61K 38/02; A61P 9/00; A61P 25/00; A61P 3/10; A61P 25/28; A61P 25/06; A61P 25/30; A61P 1/00; A61P 35/00

(52) U.S. Cl. 800/3; 435/377; 800/13; 435/320.1; 435/325; 435/7.21; 424/158.1; 514/44 R; 514/19.3; 514/1.1; 514/16.4; 514/17.7; 514/6.9; 514/18.3

(57) ABSTRACT

The present invention relates to compositions to treat glycerophosphodiester phosphodiesterase (GDE) related disorders. The invention also relates to methods treating GDE related disorders. The invention further relates to kits for treating GDE related disorders in a subject. The invention further relates to methods of identifying novel treatments for treating GDE related disorders in a subject.

### RELATED APPLICATIONS

This application claims priority to U.S. Provisional Application No. 60/721,780 filed Sep. 29, 2005, entitled, “Function of GDE Protein Family in Cellular Differentiation,” is hereby incorporated by reference in its entirety.

### GOVERNMENT SUPPORT

This work was supported by the National Institutes of Health. The government may have certain rights in the invention.

### BACKGROUND

During development of the nervous system, cell-cycle exit is coupled to cellular differentiation programs to ensure that correct numbers of neuronal subtypes are generated to construct functional neural circuits (1). This complex process involves the synchronized decrease in expression of progenitor determinants, the increase of cell-cycle inhibitors, and the implementation of defined cell fate specification programs. The molecular mechanisms that coordinate and regulate these pathways remain unclear.

### BRIEF SUMMARY OF THE INVENTION

In one aspect, provided herein are methods of modulating cellular differentiation comprising modulating the functional level of a glycerophosphodiester phosphodiesterase (GDE) protein wherein inducing over-expression of the GDE protein level or decreasing functional levels of GDE protein modulates differentiation of a cell.

In one aspect, provided herein are methods of modulating cellular differentiation in a mammal, comprising modulating the functional level of a GDE proteins wherein inducing over-expression of the GDE protein level or decreasing functional levels of GDE protein modulates differentiation of the a cell.

In one aspect, provided herein are methods for the treatment and/or prophylaxis of a condition characterized by aberrant or otherwise unwanted cellular differentiation in a mammal, comprising modulating the functional level of a GDE protein in the mammal, wherein inducing over-expression of the GDE protein level or decreasing functional levels of GDE protein modulates differentiation of the cells.

In one embodiment, the cell is one or more of a neuronal cell, a pancreatic cell, a lung cell, bone tissue cell, a spleen cell, heart cell, kidney cell, a testis cell, or an intestinal tract cell.

In another embodiment, the GDE protein comprises one or more GDE family proteins.

In a related embodiment, the GDE protein comprises glycerophosphodiester phosphodiesterase 2 (GDE2).

In one embodiment, differentiation is up-regulatable by GDE protein over-expression.

In a related embodiment, differentiation is down-regulatable by reducing the functional level of GDE protein level.

In one embodiment, the condition is one or more of cancer, degenerative diseases (ALS, Alzheimer's disease), infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), or diabetes.

In one embodiment, the modulation is up-regulation of a GDE protein level and the up-regulation comprises introducing a nucleic acid molecule encoding a GDE protein or functional equivalent, derivative or homologue thereof or the GDE protein expression product or functional derivative, homologue, analogue, equivalent or mimetic thereof to the cell.

In another embodiment, the modulation comprises contacting the cell with a compound that modulates transcriptional and/or translational regulation of a GDE gene.

In another embodiment, the modulation is up-regulation of a GDE protein level and the up-regulation comprises contacting the cell with a compound that functions as an agonist of the GDE protein expression product.

In one embodiment, the modulation is down-regulation of GDE protein levels and the down-regulation comprises contacting the cell with a compound that functions as an antagonist to the GDE protein expression product.

In one embodiment, the differentiation is modulated in vivo.

In a related embodiment, the differentiation is modulated in vitro.

In one aspect, provided herein are methods of converting a stem cell into a ventral neuron which comprises introducing into the stem cell a nucleic acid which expresses homeodomain transcription factor Nkx6.1 protein in the stem cell so as to thereby convert the stem cell into the ventral neuron.

In one aspect, provided herein are methods of converting a motor neuron progenitor into a post-mitotic neuron comprising introducing a nucleic acid expressing a GDE protein into the motor neuron progenitor to thereby convert the stem cell into the post-mitotic neuron. (not just neuron but any progenitor into its differentiated cell eg lung progenitor to differentiated lung cell)

In one embodiment, the nucleic acid incorporates into the chromosomal DNA of the cell.

In another embodiment, the nucleic acid is introduced by transfection or transduction.

In one aspect, provided herein are uses of GDE, or homologues, derivatives or fragments thereof, for the manufacture of a medicament to treat GDE related disorders.

Provided herein, according to one aspect, are pharmaceutical compositions comprising a pharmaceutically effective amount of a GDE modulator effective to treat, prevent, ameliorate, reduce or alleviate a GDE related disorder or symptoms thereof and a pharmaceutically acceptable excipient.

In one embodiment, the GDE modulator is selected from one or more of a small molecule, an anti-GDE antibody, an antigen-binding fragment of an anti-GDE antibody, a polypeptide, a peptidomimetic, a nucleic acid encoding a peptide, or an organic molecule.

In another embodiment, the GDE related disorder is pain (chronic, neuropathic, acute), trauma, migraine, neurological disorders (anxiety, stroke, psychoses, schizophrenia, depression, epilepsy), cardiovascular conditions (hypertension and cardiac arrhythmias), cancer, drug addiction, analgesic side effect, analgesic tolerance, diabetes, infertility, neurodegenerative disorders (e.g., ALS, Parkinson's, Alzheimers, spinal cord injury and/or axonal regeneration, spinal bifida (neural tube closures)) or a behavioral disorder.

Provided herein, according to one aspect, are vectors encoding one or more GDE proteins or fragments or variants thereof.

Provided herein, according to one aspect, are isolated cell that recombinantly expresses one or more peptides identified by SEQ ID NO. 1 or a message expressed from SEQ ID NO. 2, or fragments or variants thereof.

In one aspect, provided herein are methods to treat, prevent, ameliorate, reduce or alleviate a GDE related disorder or symptoms thereof, comprising: administering to a subject in need thereof a composition comprising a pharmaceutically effective amount of a GDE modulator.

In one embodiment, the GDE related disorder comprises one or more of cancer, degenerative diseases (ALS, Alzheimer's disease), infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), or diabetes.

In another embodiment, the GDE modulator is one or more of a small molecule, an anti-GDE antibody, an antigen-binding fragment of an anti-GDE antibody, a polypeptide, a peptidomimetic, a nucleic acid encoding a peptide, or an organic molecule.

In another embodiment, the GDE modulator is administered prophylactically to a subject at risk of being afflicted a GDE related disorder.

In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

In one embodiment, the composition is administered to the subject orally, intravenously, intrathecally or epidurally, intramuscularly, subcutaneously, perineurally, intradermally, topically or transcutaneously.

In another embodiment, the subject is a mammal.

In another embodiment, the subject is a human.

In one embodiment, a GDE related disorder or symptom thereof is indicated by alleviation of pain, progression of degenerative disease, fertility, reversal of nerve damage, reduction of anxiety, decreased cell proliferation, increased cell differentiation, inhibition of cell proliferation.

In another embodiment, the a GDE related disorder is one or more of cancer, degenerative diseases (ALS, Alzheimer's disease), infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), or diabetes.

In one embodiment, the methods may further comprise obtaining the GDE modulator.

In one aspect, provided herein are methods for identifying lead compounds for a pharmacological agent useful in the treatment of a GDE related disorder comprising contacting a cell expressing a GDE protein with a test compound, and measuring GDE expression or differentiation. In one embodiment, the measurement may be of the modulation (increase or decrease) of GDPD activity, e.g., glycerophosphodiesterase activity.

In one aspect, provided herein are methods for identifying lead compounds for a pharmacological agent useful in the treatment of a GDE related disorder comprising contacting a cell that does not express a functional amount of a GDE protein with a test compound, and measuring one or more of GDE expression or differentiation.

In one embodiment, GDE expression or differentiation is measured by one or more of measuring protein or RNA expression, observing physical differentiation markers, measuring protein or RNA levels of one or more of NK-homeobox 6.1, Olig2, homeobox factor 9, p27, Ngn2, islet1 or islet2.

In another embodiment, the test compounds is one or more of a peptide, a small molecule, an antibody or fragment thereof, and nucleic acid or a library thereof.

Provided herein, according to one aspect, are kits comprising an GDE modulator and a pharmaceutically acceptable carrier and b) instructions for use.

Provided herein, according to one aspect, are transgenic non-human animals comprising an over-expressed GDE protein or a fragment or variant thereof.

In one aspect, provided herein are uses of a transgenic animal as described herein to test therapeutic agents.

In one aspect, provided herein are methods for screening a therapeutic agent to treat, prevent, ameliorate, reduce or alleviate a GDE related disorder or symptoms thereof, comprising administering a test agent to a mouse having an over-expressed GDE protein, and measuring modulation of differentiation.

In another embodiment, a decrease differentiation indicates that the test agent may be useful in treating a ODE disorder. In another embodiment, changes in GDPD enzymatic activity indicate that a test agent may be useful in treating a GDE related disorder.

Other embodiments of the invention are disclosed infra.

### BRIEF DESCRIPTION OF THE DRAWINGS

FIG. 1 demonstrates GDE2 isolation and characterization. (A) Schematic depicting requirement for retinoic acid (RA) signaling at 3 distinct steps in motor neuron generation. Shh, Sonic hedgehog; FGFs, Fibroblast Growth Factors. (B) Subtractive screen to isolate retinoid-responsive genes in motor neurons. Br, brachial neural tube; FP, floorplate; ROL, retinol. (C) Reverse Northern blots showing RA-responsiveness of clone 45.1 when probed with cDNA from explants grown in the presence or absence of ROL compared with glyceraldehyde 3-phosphate dehydrogenase (GAPDH) and a non RA-responsive clone, 29.1.

FIG. 2 shows GDE2 expression in spinal motor neurons. (A-F) In situ hybridization of GDE2 mRNA in sections of chick spinal cord from limb (brachial) and non-limb (thoracic) levels. Dotted line marks the margins of the spinal cord and the motor neuron domain (MN). PM= paraxial mesoderm. (G) Schematic of molecular marker expression in ventral spinal cord. Arrow indicates medial (M) to lateral (L) axis. Dotted lines outline the intermediate zone (IZ). pMN= motor neuron progenitor domain. (H-N) Expression of GDE2 and molecular markers in HH St19 chick spinal cords. Ventral right quadrants are shown, medial is to the left, lateral to the right. Arrows in panel I, J and N respectively highlight cells which weakly stain for Nkx6.1, Olig2, or p27 and express GDE2. Dashed lines outline the margins of the spinal cord. Isll/2=Islet 1 and Islet 2; BrdU= bromodeoxyuridine, 30 minute incubation.

FIG. 3 demonstrates requirement for GDE2 in motor neuron differentiation. Right side of the spinal cord is electroporated. (A-C) GDE2 mRNA (B) and protein (C) expression after electroporation of GDE2 siRNA (D) Quantitation of HB9-expressing cells in electroporated (EP) and non-electroporated (Control) sides of the spinal cord (n=5, Student's t-test *p<0.00000001, mean+s.e.m.) (E) Quantitation of Islet2-expressing cells in electroporated (EP) and non-electroporated (Control) sides of the spinal cord (nom, Student's t-test *p=0.0000006, mean± s.e.m.).

FIG. 4 depicts premature motor neuron differentiation induced by misexpression of GDE2. Arrows mark midline of spinal cord. Right side of the spinal cord is electroporated. (A-D) HB9 expression within the ventricular zone (VZ) after electroporation of GDE2NLZ. Boxed area in C is enlarged in D. (E) Islet2 and Olig2 expression within the VZ after electroporation of GDE2NLZ. (F) Diagram of the ventral spinal cord divided into 3 bins: Bin1 and Bin2 are approximately 20 μm wide and encompass Olig2⁺ and Olig2⁺/NI1VR2⁺/HB9⁺ domains respectively. Bin3 consists predominantly of HB9⁺ and Islet2⁺ neurons. pMN= motor neuron progenitor, MN= motor neuron. (G) Number of Islet2-expressing neurons located in Bins1-3 of embryos electroporated with GDE2NLZ versus NLZ alone (mean±s.e.m., n=6). Using a Student's t-test to evaluate each pair, differences between GDE2NLZ and NLZ in Bins1 (*p<0.00000001) and 2(*p⁼0.0000004) are significant but are not in Bin3 (p=0.396). The total number of NLZ-staining cells is the same in both cases (Bin1:GDE2NLZ, 23±1; NLZ, 25±1, s.e.m., p>0.5, n=6) (H, I) Lack of BrdU incorporation by ectopic Islet2-expressing neurons generated upon GDE2NLZ electroporation. (J, K) p27 expression within the VZ after electroporation of GDE2NLZ. Dotted lines outline the spinal cord. (L-N) Islet2 expression within the VZ after electroporation of mutant GDE2H.ANLZ. Boxed area in M is enlarged in N.

FIG. 5 shows that GDE2 is expressed within or in cells directly adjacent to retinoid-rich tissues. (A-H) Serial sagittal sections of HH St25 chick embryo. (A, B) In situ hybridization showing complementary expression of GDE2 mRNA in dorsal root ganglia (DRG) and mRNA of the retinoic acid synthetic enzyme Retinaldehyde Dehydrogenase 2 (RALDH2) in surrounding paraxial mesoderm. (C, D) Complementary expression of GDE2 and RALDH2 in trachea (Tr) and lung mesenchyme (Mes). (E-F) Confocal micrographs showing GDE2 and RALDH2 proteins are localized apically in mesonephric tubules. (G-H) GDE2 and RALDH2 are expressed in cells surrounding the atrioventricular groove (AVG) and show complementary expression in the epicardial (EP) and subepicardial (SEP) layers of the heart.

FIG. 6 shows that chick GDE2 has mouse and human homologs. Alignment of GDE2 homologs from chick, c; mouse, m; and human, h. Numbers denote identity between the domains derived by CLUSTALW analysis. Shaded boxes below Roman numerals depict predicted transmembrane domains. N=N-terminus, C=C-terminus.

FIG. 7 depicts that GDE2 is oriented with the GDPD domain located extracellularly and the N- and C-termini located intracellularly. (A, B) HEK293 cells transfected with GDE2NLZ. Visualization of GDE2 by the polyclonal antibody raised against the C-terminus is only possible upon addition of detergent to permeabilize the cells. (C, D) HEK293 cells transfected with N-terminal FLAG-tagged GDE2 and (E, F) C-terminal FLAG-tagged GDE2. Visualization of the tagged proteins is only possible after permeabilization suggesting that the N- and C-termini of GDE2 are located intracellularly. (G, H) HEK293 cells transfected with GDE2 modified to include a FLAG tag on the GDPD domain (GDE2-GDPD-FLAG). The FLAG epitope can be detected without permeabilization indicating that the GDPD domain is oriented extracellularly. TO-PRO3 staining marks cell nuclei.

FIG. 8 depicts that siRNA-mediated silencing of GDE2 is sequence-specific. (A-D) In situ hybridization of St21 chick embryos for GDE2 mRNA. The right side of the spinal cord is electroporated in all cases (A) Electroporation of 1.75 μg/p.l (1× dose) GDE2 siRNA results in significant GDE2 loss (B) Electroporation of 0.8751 .tg/μl (0.5× dose) GDE2 siRNA results in a smaller decrease in GDE2 expression (C, D) Two different examples of embryos electroporated with 1.75 μg/μl of siRNA targeting DsRED show no loss of GDE2.

FIG. 9 shows electroporation of GDE2 siRNA leads to a loss of motor neurons but no change in motor neuron “progenitors or dorsal-ventral patterning of the spinal cord. Right side of the spinal cord is electroporated. (A-C) Expression of the motor neuron markers MNR2/HB9, Islet', and Islet2 in embryos electroporated with GDE2 siRNA. (D-G) Islet2 and Olig2 expression in an embryo electroporated with GDE2 siRNA. The number of Olig2-expressing cells is unaffected by GDE2 siRNA electroporation (n⁼6, Student's t-test p>0.5, mean+s.e.m.). (H-K) Olig2, Pax6, Nkx6.1, and Nkx2.2 expression in HE St21 chick spinal cords after electroporation with GDE2 siRNA (n=4-6 embryos).

FIG. 10 depicts that GDPD domains share homology with the PI-PLC catalytic domain. Alignment of N-terminal catalytic-X-domain sequences of human (h) PI-PLC subtypes with C-terminal GDPD sequences of rat (r) GDE1 and chick (c) GDE2. Con-Consensus sequence for PI-PLC. Green, blue, black= identical, conserved change, non-conserved amino-acid residues. *= residues essential for catalytic activity in PI-PLC.

FIG. 11 shows that wild-type and mutant GDE2 both localize to the plasma membrane. (A) HEK293 cells transfected with GDE2NLZ and (B) GDE2H.ANLZ both show plasma membrane localization of GDE2 protein and nuclear staining of LacZ. Cells expressing very high levels of LacZ show some expression of GDE2 within the cytosol in both cases. TO-PRO3 marks cell nuclei.

FIG. 12 shows that GDE2 can act non cell-autonomously over short ranges. (A-D) HB9 and NLZ expression in the spinal cord after electroporation of GDE2NLZ. Arrows mark examples of cells in the VZ of the electroporated side of the spinal cord that express HB9 but do not express NLZ. Dotted lines indicate the midline of the spinal cord. In all cases, NLZ expression is coincident with GDE2 misexpression. (E) Quantitation of the percentage of HB9-expressing cells in Bin 1 (FIG. 4F) that also express NLZ (n=5, mean±s.e.m., *p=0.0000000032).

FIG. 13 is an amino acid sequence of GDE2.

FIG. 14 is a nucleotide sequence of GDE2.

### DETAILED DESCRIPTION

This invention is based, in part, on the discovery of that the GDE protein, GDE2, is necessary and sufficient to drive differentiation of cells. The present invention provides novel compositions, methods, and kits to treat a GDE related disorders. The invention further provides methods of identifying novel treatments for treating GDE related disorders in a subject.

Definitions

“Agonist,” as used herein refers to a compound or composition capable of combining with (e.g., binding to, interacting with) receptors to initiate pharmacological actions.

Pharmaceutically acceptable refers to, for example, compounds, materials, compositions, and/or dosage forms which are suitable for use in contact with the tissues of human beings and animals without excessive toxicity, irritation, allergic response, or other problems or complications, commensurate with a reasonable benefit/risk ratio.

Pharmaceutically acceptable salts refer to, for example, derivatives of the disclosed compounds wherein the compounds are modified by making at least one acid or base salt thereof, and includes inorganic and organic salts.

An effective antagonistic amount of GDE modulator refers to an amount that effectively attenuates (e.g. blocks, inhibits, prevents, or competes with) the activity of the GDE protein.

A therapeutically effective amount of a GDE composition refers to an amount that elicits alleviation or lessening of at least one symptom of pain upon administration to a subject in need thereof.

Potency refer, for example, to the strength of a composition or treatment in producing desired effects, for example, differentiation and/or the alleviation of, for example, symptoms described infra. Potency also may refer to the effectiveness or efficacy of a composition in eliciting desired effects, for example, initiation of differentiation or exit from differentiation. Enhanced potency, for example, refers to the lowering of a dose in achieving desired effects or to an increased therapeutic benefit including that not previously seen, for example, where the increased therapeutic benefit is eliciting desired effects such as differentiation from oral administration, oral formulation or oral dosage form. In therapeutics, for example, potency may refer to the relative pharmacological activity of a compound or a composition.

The following terms encompass polypeptides that are identified in Genbank by the following designations, as well as polypeptides that are at least about 70% identical to polypeptides identified in Genbank by these designations as described infra. In alternative embodiments, these terms encompass polypeptides identified in Genbank by these designations and polypeptides sharing at least about 80, 90, 95, 96, 97, 98, or 99% identity.

A “GDE modulator” is either an inhibitor or an enhancer of a GDE protein family member. A “non-selective” GDE protein family modulator is an agent that modulates other GDE protein family members at the concentrations typically employed for GDE modulation. A “selective” GDE modulator significantly modulates one or more of the normal functions of an GDE protein family member at a concentration at which other GDE proteins are not significantly modulated. A modulator “acts directly on” a GDE protein family member when the modulator binds to the GDE protein. A modulator “acts indirectly on a GDE protein” when the modulator binds to a molecule other than the GDE protein, which binding results in modulation of the protein.

A “modulator of a GED protein” is an agent that reduces, by any mechanism, the extent of depolarization-induced inward current through GDE protein family members, as compared to that observed in the absence (or presence of a smaller amount) of the agent. A modulator of a. GDE protein can affect: (1) the expression; mRNA stability; or protein trafficking, modification (e.g., phosphorylation), or degradation of a GDE protein family member, or (2) one or more of the normal functions of a GDE protein family member, such the depolarization-induced inward current. An modulator of a GDE protein family member can be non-selective or selective.

An “enhancer of a GDE protein” is an agent that increases, by any mechanism as compared to that observed in the absence (or presence of a smaller amount) of the agent. An enhancer of a GDE protein can affect: (1) the expression; mRNA stability; or protein trafficking, modification (e.g., phosphorylation), or degradation of a GDE protein; or (2) one or more of the normal functions of a GDE protein. An enhancer of an GDE protein can be non-selective or selective.

In one embodiment the present invention is directed to up regulating the functional level of ODE to introducing differentiation to a population of cells. However, it should nevertheless be understood that there are circumstances in which it is desirable to down regulate the functional level of GDE to obviate the expression of these characteristics or to end aberrant differentiation. For example, one may seek to up regulate the functional level of GDE in the context of a defined population of cells for a period of time sufficient to achieve a particular objective. However, once that objective has been achieved one would likely seek to down regulate the intracellular functional level of GDE, to the extent that it is not transient, such that it is no longer over-expressed and the subject cells. In another example, one may identify certain disease conditions which are characterized by an over-expression of the functional level of GDE, e.g., cancer. In such a situation, one may observe uncontrolled cell proliferation which could lead to tumor formation. Where such a situation exists, one may seek to down regulate the functional level of GDE to end aberrant differentiation. Accordingly, down-regulation of cell GDE levels would be desirable as a therapeutic treatment. The present invention should therefore be understood to be directed to up regulating the GDE functional level in order to introduce unique phenotypic properties to the population of cells and down-regulating a naturally or non-naturally induced state of GDE over-expression.

As detailed above, reference to “modulating” GDE functional levels is a reference to either up regulating or down regulating these levels. Such modulation may be achieved by any suitable means and include, for example: (i) modulating absolute levels of the active or inactive forms of GDE (for example increasing or decreasing intracellular GDE concentrations) such that either more or less GDE is available for activation and/or to interact with its downstream targets. (ii) Agonising or antagonising GDE such that the functional effectiveness of any given GDE molecule is either increased or decreased. For example, increasing the half life of GDE may achieve an increase in the overall level of GDE activity without actually necessitating an increase in the absolute intracellular concentration of GDE. Similarly, the partial antagonism of GDE, for example by coupling GDE to a molecule that introduces some steric hindrance in relation to the binding of GDE to its downstream targets, may act to reduce, although not necessarily eliminate, the effectiveness of GDE signaling. Accordingly, this may provide a means of down-regulating GDE functioning without necessarily down-regulating absolute concentrations of GDE.

In terms of achieving the up or down-regulation of GDE functioning, methods and techniques for achieving this objective would be well known to the person of skill in the art and include, for example: (i) introducing into a cell a nucleic acid molecule encoding GDE or functional equivalent, derivative or analogue thereof in order to up-regulate the capacity of The cell to express GDE. (ii) Introducing into a cell a proteinaceous or non-proteinaceous molecule which modulates transcriptional and/or translational regulation of a gene, wherein this gene may be a GDE gene or functional portion thereof or some other gene which directly or indirectly modulates the expression of the GDE gene. (iii) introducing into a cell the GDE expression product (in either active or inactive form) or a functional derivative, homologue, analogue, equivalent or mimetic thereof. (iv) introducing a proteinaceous or non-proteinaceous molecule which functions as an antagonist to the GDE expression product. (v) introducing a proteinaceous or non-proteinaceous molecule which functions as an agonist of the GDE expression product.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Anti-inflammatory compounds directed at blocking or reducing synovial inflammation, and thereby improving function, and analgesics directed to reducing pain, are presently the primary method of treating the rheumatoid diseases and arthritis. Aspirin and other salicylate compounds are frequently used in treatment to interrupt amplification of the inflammatory process and temporarily relieve the pain. Other drug compounds used for these purposes include phenylpropionic acid derivatives such as Ibuprofen and Naproxin, Sulindac, phenyl butazone, corticosteroids, antimalarials such as chloroquine and hydroxychloroquine sulfate, and fenemates. For a thorough review of various drugs utilized in treating rheumatic diseases, reference is made to J. Hosp. Pharm., 36:622 (May 1979).

Neurological disorders include, for example, disorders involving the brain, cortex, dorsal root ganglion (DRG) neurons, sciatic nerve, and spinal cord.

Disorders involving the brain include, for example, disorders involving neurons, and disorders involving glia, and developmental diseases, such as neural tube defects, forebrain anomalies, posterior fossa anomalies, and syringomyelia and hydromyelia; perinatal brain injury; cerebrovascular diseases; infections, such as acute meningitis, acute focal suppurative infections, including brain abscess, subdural empyema, and extradural abscess, chronic bacterial meningoencephalitis, including tuberculosis and mycobacterioses, neurosyphilis, and neuroborreliosis, viral meningoencephalitis, progressive multifocal leukoencephalopathy, subacute sclerosing panencephalitis; fungal meningoencephalitis; transmissible spongiform encephalopathies; demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, spinocerebellar degenerations; inborn errors of metabolism; and toxic and acquired metabolic diseases. Disorders of the peripheral nervous system include, inflammatory neuropathies, such as, immune-mediated neuropathies; infectious polyneuropathies, such as, leprosy, diphtheria, varicella-zoster virus; hereditary neuropathies, such as, hereditary motor and sensory neuropathy I, HMSN II, Dejerine-Sottas Disease; acquired metabolic and toxic neuropathies, such as, peripheral neuropathy in adult-onset diabetes mellitus, metabolic and nutritional peripheral neuropathies, neuropathies associated with malignancy, toxic neuropathies; traumatic neuropathies; and tumors of the peripheral nerve.

A “test agent” is any agent that can be screened in the prescreening or screening assays of the invention. The test agent can be any suitable composition, including a small molecule, peptide, or polypeptide.

The term “therapy,” as used herein, encompasses the treatment of an existing condition as well as preventative treatment (i.e., prophylaxis). Accordingly, “therapeutic” effects and applications include prophylactic effects and applications, respectively.

A used herein, the term “high risk” refers to an elevated risk as compared to that of an appropriate matched (e.g., for age, sex, etc.) control population.

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is often less labile than RNA. Similarly, chemical modifications that enhance an aptamer's specificity or stability are preferred.

Chemical modifications that may be incorporated into nucleic acids include, with neither limitation nor exclusivity, base modifications, sugar modifications, and backbone modifications. Base modifications: The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenylade-nine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers. Sugar modifications: The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and H is; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3-(and 4-) hydroxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Om), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.go-v/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

A “radioligand binding assay” is an assay in which a biological sample (e.g., cell, cell lysate, tissue, etc.) containing a receptor is contacted with a radioactively labeled ligand for the receptor under conditions suitable for specific binding between the receptor and ligand, unbound ligand is removed, and receptor binding is determined by detecting bound radioactivity.

As used herein, an “antibody” refers to a protein consisting of one or more polypeptides substantially encoded by immunoglobulin genes or fragments of immunoglobulin genes. Immunoglobulin genes include, for example, the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term antibody, as used herein also includes antibody fragments either produced by the modification of whole antibodies or synthesized de novo using recombinant DNA methodologies, see for example, Fundamental Immunology, W. E. Paul, ed., Raven Press, N.Y. (1993), for a more detailed description of other antibody fragments. While various antibody fragments are defined in terms of the digestion of an intact antibody, one of skill will appreciate that such Fab′ fragments may be synthesized de novo either chemically or by utilizing recombinant DNA methodology. Antibodies also include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The phrases “an effective amount” and “an amount sufficient to” refer to amounts of a biologically active agent that produce an intended biological activity.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH 7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“GDE” or “GDE protein” or GDE protein family” refer to a protein or family of glycerophosphodiester phosphodiesterase proteins. These protein contain a glycerophosphodiester phosphodiesterase (GDPD) domain.

It has also been determined that expressing or over-expressing GDE in a cell can result in the induction of differentiation. Accordingly, reference to “modulating” differentiation of a cell “relative to” normal cell characteristics should be understood to include the over-expression of GDE levels results in the induction of differentiation that is not generally observed in the context of cells that do not express GDE at a functional level. Without limiting the present invention in any way, examples of characteristics which may be induced in cells over-expressing GDE levels include, for example:

1) improved proliferative characteristics both in terms of an increased rate/extent of proliferation and the requirement for only minimal environmental/cell culture conditions under which proliferation can occur (herein referred to as “enhanced proliferation”);

2) improved cell viability, which may occur either at the level of down regulating apoptosis or preventing or otherwise induced cell death. For example, cell survival under conditions of stress (such as the removal of tissue culture supplements in the in vitro environment) is facilitated as is the down regulation of apoptosis which would normally occur in the absence of the anti-apoptotic signals which are provided as a result of integrin receptor engagement during matrix attachment and cell spreading. This is particularly relevant, for example, where in vitro cell culture populations are required to be maintained in suspension (herein referred to as “enhanced viability”); or

3) changed differentiation pathways.

As used herein, “functional level” of GDE should be understood as a reference to the level of GDE activity which is present in any given cell as opposed to the concentration of GDE. Although an increase in the concentration of GDE will generally correlate to an increase in the level of GDE functional activity which is observed in a cell, the person skilled in the art would also understand that increases in the level of activity can be achieved by means other than merely increasing absolute intracellular GDE concentrations. For example, one might utilize forms of GDE which exhibit an increased half-life or otherwise exhibit enhanced activity. Reference to “over-expressing” the subject GDE level should therefore be understood as a reference to up regulating intracellular GDE to an effective functional level which is greater than that expressed under the normal physiological conditions for a given cell prior to differentiation or to the up-regulation of GDE levels to any level of functionality but where that up-regulation event is one which is artificially effected rather than being an increase which has occurred in the subject cell due to the effects of naturally occurring physiology prior to differentiation. Accordingly, this latter form of up-regulation may correlate to up-regulating GDE to levels which fall within the normal physiological range but which are higher than pre-stimulation or pre-differentiation levels. The mechanism by which up-regulation is achieved may be artificial mechanism that seek to mimic a physiological pathway—for example introducing a hormone or other stimulatory molecule, e.g., retinoic acid (RA). Accordingly, the term “expressing” is not intended to be limited to the notion of GDE gene transcription and translation. Rather, it is a reference to an outcome, being the establishment of a higher and effective functional level of GDE than is found under normal physiological conditions in a cell at a particular point in time (e.g., it includes non-naturally occurring increases in GDE level, even where those increases may fall within the normal physiological range which one might observe). Reference to the subject functional level being an “effective” level should be understood as a level of over-expression which achieves the modulation of differentiation of a cell relative to a normal cell.

Reference to “modulating” in the context of cell differentiation includes, for example, inducing the differentiation. In the context of the functional level of GDE, reference to “modulating” includes, for example, up regulating or down regulating the functional level of GDE. Determining the specific functional level (e.g., “effective” level) to which the GDE should be up or down-regulated in order to achieve the desired phenotypic change for any given cell type is a matter of routine procedure. The person of skill in the art would be familiar with methods of determining such a level. “Modulating cellular differentiation,” as used herein includes, any up or down-regulation of differentiation. It also includes initiation or advancing the stage of differentiation or the exiting of differentiation.

Methods of Treating

In one aspect, provided herein are methods to treat, prevent, ameliorate, reduce or alleviate a GDE related disorder or symptoms thereof, comprising: administering to a subject in need thereof a composition comprising a pharmaceutically effective amount of a GDE modulator.

An “effective amount” includes, for example, an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

In one embodiment, the composition is administered to the subject orally, intravenously, intrathecally or epidurally, intramuscularly, subcutaneously, perineurally, intradermally, topically or transcutaneously.

Subjects include mammals, e.g., humans, cows, pigs, horses, squirrels, primates, dogs, cats, rabbits, goats, etc.

“Obtaining the GDE modulator,” as used herein refers to making or buying the modulator.

In one embodiment, a GDE related disorder or symptom thereof is indicated by alleviation of pain, progression of degenerative disease, fertility, reversal of nerve damage, reduction of anxiety, decreased cell proliferation, increased cell differentiation, or inhibition of cell proliferation.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered to include reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, for example, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in herein may be co-administered with one or more other compounds or molecules. By “co-administered” is meant simultaneous or sequential administration in the same formulation or in two different formulations via the same or different routes or sequential administration by the same or different routes. For example, the subject GDE may be administered together with an agonistic agent in order to enhance its effects. Alternatively, in the case of organ tissue transplantation, the GDE may be administered together with immunosuppressive drugs. By “sequential” administration is meant a time difference of from seconds, minutes, hours or days between the administration of the two types of molecules. These molecules may be administered in any order. In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

Another aspect of the present invention relates to the use of an agent capable of modulating the functional level of GDE in the manufacture of a medicament for the modulation of cell differentiation in a mammal wherein inducing over-expression of the GDE level modulates cell differentiation of the cells.

In another aspect, the present invention relates to the use of GDE or a nucleic acid encoding GDE in the manufacture of a medicament for the modulation of cell differentiation in a mammal wherein inducing over-expression of the GDE level modulates cell differentiation of the cells.

“Aberrant or otherwise unwanted cellular differentiation” refers, for example, to conditions in a mammal, wherein differentiation desired and not occurring or vice verse.

Aberrant differentiation may happen, for example, one or more of a neuronal cell, a pancreatic cell, a lung cell, bone tissue cell, a spleen cell, heart cell, kidney cell, a testis cell, or an intestinal tract cell. The aberrant differentiation may lead, for example, to one or more of the following conditions: cancer, degenerative diseases (ALS, Alzheimer's disease), infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), or diabetes. The differentiation is up-regulatable by GDE protein over-expression and down-regulatable by reducing the functional level of GDE protein level.

The modulation may be the up-regulation of a GDE protein level and the up-regulation for example by the introduction a nucleic acid molecule encoding a GDE protein or functional equivalent, derivative or homologue thereof or the GDE protein expression product or functional derivative, homologue, analogue, equivalent or mimetic thereof to the cell. The modulation may also be by contacting the cell with a compound that modulates transcriptional and/or translational regulation of a GDE gene. The modulation may also be by contacting the cell with a compound that functions as an agonist of the GDE protein expression product.

In the one embodiment, the modulation is down-regulation of GDE protein levels and the down-regulation may be done by contacting the cell with a compound that functions as an antagonist to the GDE protein expression product.

In either up- or down-regulation, the modulation of differentiation may be in vivo or in vitro.

In one aspect, provided herein are methods of converting a stem cell into a ventral neuron which comprises introducing into the stem cell a nucleic acid which expresses homeodomain transcription factor Nkx6.1 protein in the stem cell so as to thereby convert the stein cell into the ventral neuron.

In one aspect, provided herein are methods of converting a motor neuron progenitor into a post-mitotic neuron comprising introducing a nucleic acid expressing a GDE protein into the motor neuron progenitor to thereby convert the stem cell into the post-mitotic neuron or any progenitor into its differentiated cell, e.g., lung progenitor to differentiated lung cell.

In one aspect, provided herein are methods of converting progenitor cell into a differentiated cell (e.g., a lung progenitor into a lung cell) comprising introducing a nucleic acid expressing a GDE protein into the progenitor to thereby convert the stem cell into the differentiated cell.

In certain methods, nucleic acid incorporates into the chromosomal DNA of the cell. For example, the DNA may be introduced by transfection or transduction and other methods known to the skilled artisan.

In one aspect, provided herein are uses of GDE, or homologues, derivatives or fragments thereof, for the manufacture of a medicament to treat GDE related disorders.

Provided herein, according to one aspect, are pharmaceutical compositions comprising a pharmaceutically effective amount of a GDE modulator effective to treat, prevent, ameliorate, reduce or alleviate a GDE related disorder or symptoms thereof and a pharmaceutically acceptable excipient.

In one embodiment, the GDE modulator is selected from one or more of a small molecule, an anti-GDE antibody, an antigen-binding fragment of an anti-GDE antibody, a polypeptide, a peptidomimetic, a nucleic acid encoding a peptide, or an organic molecule.

In another embodiment, the GDE related disorder is cancer, infertility, pulmonary disease, tissue engineering, nerve damage, gastrointestinal disease, pain (chronic, neuropathic, acute), trauma, migraine, neurological disorders (anxiety, stroke, psychoses, schizophrenia, depression, epilepsy), cardiovascular conditions (hypertension and cardiac arrhythmias), diabetes, cancer, drug addiction, analgesic side effect, analgesic tolerance, diabetes, infertility, neurodegenerative disorders (e.g., ALS, Parkinson's. Alzheimers, spinal cord injury and axonal regeneration, spinal bifida (neural tube closures)) or a behavioral disorder.

The present invention provides for both prophylactic and therapeutic methods of treating a subject at risk of, or susceptible to, a GDE related disease or disorder. Treatment is defined as the application or administration of a therapeutic agent to a patient, or application or administration of a therapeutic agent to an isolated tissue or cell line from a patient, who has a GDE related disease or disorder, a symptom of a GDE related disease or disorder or a predisposition toward a GDE related disease or disorder, with the purpose to cure, heal, alleviate, relieve, alter, remedy, ameliorate, improve or affect the disease or disorder, the symptoms of the disease or disorder or the predisposition toward the disease or disorder.

The therapeutic methods of the invention involve the administration of the polypeptide and/or nucleic acid molecules of the invention as described herein.

In one aspect, the invention provides a method for preventing a GDE related disease or disorder in a subject by administering to the subject a polypeptide or nucleic acid molecule of the invention as described herein.

The invention provides therapeutic methods and compositions for the prevention and treatment of a GDE realted disease or disorder. In particular, the invention provides methods and compositions for the prevention and treatment of the disease or disorder in subjects.

In one embodiment, the present invention contemplates a method of treatment, comprising: a) providing, i.e., administering: i) a mammalian patient particularly human who has, or is at risk of developing a GDE disease or disorder, one or more molecules of the invention as described herein.

The term “at risk for developing” is herein defined as individuals an increased probability of contracting an GDE realted disease or disorder due to exposure or other health factors.

The present invention is also not limited by the degree of benefit achieved by the administration of the molecule. For example, the present invention is not limited to circumstances where all symptoms are eliminated. In one embodiment, administering a molecule reduces the number or severity of symptoms of a GDE related disease or disorder. In another embodiment, administering of a molecule may delay the onset of symptoms of a GDE related disease or disorder.

Yet another aspect of this invention relates to a method of treating a subject (e.g., mammal, human, horse, dog, cat, mouse) having a disease or disease symptom (including, but not limited to angina, hypertension, congestive heart failure, myocardial ischemia, arrhythmia, diabetes, urinary incontinence, stroke, pain, traumatic brain injury, or a neuronal disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g., opinion) or objective (e.g., measurable by a test or diagnostic method).

Typical subjects for treatment in accordance with the individuals include mammals, such as primates, preferably humans. Cells treated in accordance with the invention also preferably are mammalian, particularly primate, especially human. As discussed above, a subject or cells are suitably identified as in needed of treatment, and the identified cells or subject are then selected for treatment and administered one or more of fusion molecules of the invention.

The treatment methods and compositions of the invention also will be useful for treatment of mammals other than humans, including for veterinary applications such as to treat horses and livestock e.g., cattle, sheep, cows, goats, swine and the like, and pets such as dogs and cats.

In other embodiments, the inhibition GDE protein family members can be achieved by any available means, e.g., inhibition of (1) the expression, mRNA stability, protein trafficking, modification (e.g., phosphorylation), or degradation of an GDE protein family member, or (2) one or more of the normal functions of an GDE protein family member.

In one embodiment, GDE protein family member inhibition is achieved by reducing the level of GDE protein family members in a tissue expressing the protein. Thus, the method of the invention can target GDE protein family members in tissues wherein the protein is expressed as described infra. This can be achieved using, e.g., antisense or RNA interference (RNAi) techniques to reduce the level of the RNA available for translation.

Methods of Screening

The role of GDE protein family members in mediating a GDE related disorders makes the GDE protein family member an attractive target for agents that modulate these disorders to effectively treat, prevent, ameliorate, reduce or alleviate the disorders. Accordingly, the invention provides prescreening and screening methods aimed at identifying such agents. The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof or in animals.

In one embodiment, therefore, a prescreening method comprises contacting a test agent with an GDE protein family member. Such prescreening is generally most conveniently accomplished with a simple in vitro binding assay. Means of assaying for specific binding of a test agent to a polypeptide are well known to those of skill in the art and are detailed in the Examples infra. In one binding assay, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various screening formats are discussed in greater detail below.

Test agents, including, for example, those identified in a prescreening assay of the invention can also be screened to determine whether the test agent affects the levels of GDE protein family members or RNA. Agents that reduce these levels can potentially reduce one or more GDE related disorders.

Accordingly, the invention provides a method of screening for an agent that modulates a GDE related disorder in which a test agent is contacted with a cell that expresses a GDE protein family member in the absence of test agent. Preferably, the method is carried out using an in vitro assay or in vivo. In such assays, the test agent can be contacted with a cell in culture or to a tissue. Alternatively, the test agent can be contacted with a cell lysate or fraction thereof (e.g., a membrane fraction for detection of GDE protein family members or polypeptides thereof). The level of (i) GDE protein family members; or RNA is determined in the presence and absence (or presence of a lower amount) of test agent to identify any test agents that alter the level. If the level assayed is altered, the test agent is selected as a potential modulator of a GDE related disorder. In a preferred embodiment, an agent that reduces or increases the level assayed is selected as a potential modulator of one or more GDE related disorders.

Cells useful in this screening method include those from any of the species described above in connection with the method of reducing a drug-related effect or behavior. Cells that naturally express an GDE protein family member are useful in this screening methods. Examples include PC12 cells, SH-SY5y cells, NG108-15 cells, IMR-32 cells, SK-N-SH cells, RINm5F cells, and MB cells. Alternatively, cells that have been engineered to express a GDE protein family member can be used in the method.

In one embodiment, the test agent is contacted with the cell in the presence of a drug. The drug is generally one that produces one or more undesirable effects or behaviors, such as, for example, sedative-hypnotic and analgesic drugs. In particular embodiments, the drug is ethanol, a cannabinioid, or an opioid.

As noted above, screening assays are generally carried out in vitro, for example, in cultured cells, in a biological sample (e.g., brain, dorsal root ganglion neurons, and sympathetic ganglion neurons), or fractions thereof. For ease of description, cell cultures, biological samples, and fractions are referred to as “samples” below. The sample is generally derived from an animal (e.g., any of the research animals mentioned above), preferably a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

GDE protein family members can be detected and quantified by any of a number of methods well known to those of skill in the art. Examples of analytic biochemical methods suitable for detecting GDE protein family member, include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), receptor-linked immunosorbent assays (ELISAs), immunofluorescent assays, Western blotting, fluorescence resonance energy transfer (FRET) assays, yeast two-hybrid assays, whole or partial cell current recordings, and the like. Peptide modulators may be discovered or screened for example, by phage display. See 5,096,815; 5,198,346; 5,223,409; 5,260,203; 5,403,484; 5,534,621; and 5,571,698.

Methods for identifying lead compounds for a pharmacological agent useful in the treatment of a GDE related disorder comprising contacting a GDE protein with a test compound, and measuring differentiation. The GDE protein may also be a modified, e.g., a chimeric and/or a deletion mutant. The GDE protein may be isolated or may be in a membrane or an artificial membrane. The contacting may be directly or indirectly.

Methods of the invention also include methods for screening a therapeutic agent to treat, prevent, ameliorate, reduce or alleviate a GDE related disorder or symptoms thereof, comprising administering a test agent to a mouse having an over-expressed GDE protein.

The proteinaceous molecules described above may be derived from any suitable source such as natural, recombinant or synthetic sources and includes fusion proteins or molecules which have been identified following, for example, natural product screening or high-throughput screening. The reference to non-proteinaceous molecules may be, for example, a reference to a nucleic acid molecule or it may be a molecule derived from natural sources, such as for example natural product screening, or may be a chemically synthesized molecule. The present invention contemplates analogues of the GDE expression product or small molecules capable of acting as agonists or antagonists. Chemical agonists may not necessarily be derived from the GDE expression product but may share certain conformational similarities. Alternatively, chemical agonists may be specifically designed to meet certain physiochemical properties. Antagonists may be any compound capable of blocking, inhibiting or otherwise preventing GDE from carrying out its normal biological function, such as molecules which prevent its activation or else prevent the downstream functioning of activated GDE. Antagonists include monoclonal antibodies and antisense nucleic acids which prevent transcription or translation of GDE genes or mRNA in mammalian cells. Modulation of expression may also be achieved utilizing antigens, RNA, ribosomes, DNAzymes, RNA aptamers, antibodies or molecules suitable for use in co-suppression. The proteinaceous and non-proteinaceous molecules referred to in points (i)-(v), above, are herein collectively referred to as “modulatory agents”. In another embodiment, the GDE modulator is one or more of a small molecule, an anti-GDE antibody, an antigen-binding fragment of an anti-ODE antibody, a polypeptide, a peptidomimetic, a nucleic acid encoding a peptide, or an organic molecule.

Screening for the modulatory agents can be achieved by any one of several suitable methods including, but in no way limited to, contacting a cell comprising the GDE gene or functional equivalent or derivative thereof with an agent and screening for the modulation of GDE protein production or functional activity, modulation of the expression of a nucleic acid molecule encoding GDE or modulation of the activity or expression of a downstream GDE cellular target. Detecting such modulation can be achieved utilizing techniques such as Western blotting, electrophoretic mobility shift assays and/or the readout of reporters of GDE activity such as luciferases, CAT and the like or observation of morphological changes.

The GDE gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate GDE activity, at either the nucleic acid or expression product levels, or the gene may require activation—thereby providing a model useful for, inter alia, screening for agents which up regulate GDE expression. Further, to the extent that a GDE nucleic acid molecule is transfected into a cell, that molecule may comprise the entire GDE gene or it may merely comprise a portion of the gene such as the portion which regulates expression of the GDE product. For example, the GDE promoter region may be transfected into the cell which is the subject of testing. In this regard, where only the promoter is utilized, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream GDE regulatory target, rather than GDE itself. Yet another example includes GDE binding sites ligated to a minimal reporter. For example, modulation of GDE activity can be detected by screening for the modulation of the functional activity in a cell. This is an example of an indirect system where modulation of GDE expression, per se, is not the subject of detection. Rather, modulation of the molecules which GDE regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the GDE nucleic acid molecule or expression product itself or which modulate the expression of an upstream molecule, which upstream molecule subsequently modulates GDE expression or expression product activity. Accordingly, these methods provide a mechanism for detecting agents which either directly or indirectly modulate GDE expression and/or activity.

The agents which are utilized in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, The agent is associated with a molecule which permits its targeting to a localized region.

The proteinaceous or non-proteinaceous molecules may act either directly or indirectly to modulate the expression of GDE or the activity of the GDE expression product. The molecule acts directly if it associates with the GDE nucleic acid molecule or expression product to modulate expression or activity, respectively. The molecule acts indirectly if it associates with a molecule other than the GDE nucleic acid molecule or expression product which other molecule either directly or indirectly modulates the expression or activity of the GDE nucleic acid molecule or expression product, respectively. Accordingly, the method of the present invention encompasses the regulation of GDE nucleic acid molecule expression or expression product activity via the induction of a cascade of regulatory steps.

The term “expression” refers, for example, to the transcription and translation of a nucleic acid molecule. Reference to “expression product” is a reference to the product produced from the transcription and translation of a nucleic acid molecule.

“Derivatives” of the molecules herein described (for example GDE or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, GDE or derivative thereof may be fused to a molecule to facilitate its entry into a cell. Analogues of the molecules contemplated herein include, for example, modification to side chains, incorporating of unnatural amino acids and/or their derivatives during peptide, polypeptide or protein synthesis and the use of crosslinkers and other methods including conformational constraints on the proteinaceous molecules or their analogues.

Derivatives of nucleic acid sequences which may be utilized in accordance with the method described herein may similarly be derived from single or multiple nucleotide substitutions, deletions and/or additions including fusion with other nucleic acid molecules. The derivatives of the nucleic acid molecules utilized as described herein include, for example, oligonucleotides, PCR primers, antisense molecules, molecules suitable for use in co-suppression and fusion of nucleic acid molecules. Derivatives of nucleic acid sequences also include degenerate variants.

A “variant” of GDE should be understood to include, for example, molecules that exhibit at least some of the functional activity of the form of GDE of which it is a variant. A variation may take any form and may be naturally or non-naturally occurring. A mutant molecule is one which exhibits, for example, modified functional activity.

A “homologue” is includes, for example, that the molecule is derived from a species other than that which is being treated in accordance with the method of the present invention. This may occur, for example, where it is determined that a species other than that which is being treated produces a form of GDE which exhibits similar and suitable differentiation to that of the GDE which is naturally produced by the subject undergoing treatment.

Chemical and functional equivalents include, for example, molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

In one aspect, provided herein are methods for screening a therapeutic agent to treat, prevent, ameliorate, reduce or alleviate a GDE related disorder or symptoms thereof, comprising administering a test agent to a mouse having an over-expressed GDE protein, and measuring modulation of differentiation. In one aspect, provided herein are methods for identifying lead compounds for a pharmacological agent useful in the treatment of a GDE related disorder comprising contacting a cell expressing a GDE protein with a test compound, and measuring GDE expression, modulation, or differentiation or modulation of GDPD activity (e.g., glycerophosphodiesterase activity).

In one aspect, provided herein are methods for identifying lead compounds for a pharmacological agent useful in the treatment of a GDE related disorder comprising contacting a cell that does not express a functional amount of a GDE protein with a test compound, and measuring one or more of GDE expression or differentiation.

In one embodiment, GDE expression or differentiation is measured by one or more of measuring protein or RNA expression, observing physical differentiation markers, measuring protein or RNA levels of one or more of NK-homeobox 6.1 (Gen Bank Assession No. NP_{—}796374), Olig2 (Gen Bank Assession Nos.: AAH36245; BAB18907; NP_{—}005797; Q9EQW6), homeobox factor 9 (Gen Bank Assession Nos.: NP_{—}064328; NP_{—}005506; P50219; Q9QZW9), p27 (Gen Bank Assession Nos.: BAA25263; NP_{—}034005;), Ngn2 (Gen Bank Assession Nos.: NP_{—}033848; Q9H2A3; NP_{—}076924; AAH36847), islet1 (Gen Bank Assession No.: NP_{—}002193) or islet2 (Gen Bank Assession No.: NP_{—}081673).

In another embodiment, the test compounds is one or more of a peptide, a small molecule, an antibody or fragment thereof, and nucleic acid or a library thereof.

Also useful in the screening techniques described herein are combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing GDE analogues which exhibit properties such as more potent pharmacological effects.

With respect to high throughput library screening methods, oligomeric or small-molecule library compounds capable of interacting specifically with a selected biological agent, such as a biomolecule, a macromolecule complex, or cell, are screened utilizing a combinational library device which is easily chosen by the person of skill in the art from the range of well-known methods, such as those described above. In such a method, each member of the library is screened for its ability to interact specifically with the selected agent. In practicing the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of GDE, it may also be desirable to identify and utilize molecules which function agonistically or antagonistically to GDE in order to up or down-regulate the functional activity of GDE in relation to modulating cell differentiation. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of GDE capable of acting as agonists or antagonists. Chemical agonists may not necessarily be derived from GDE but may share certain conformational similarities. Alternatively, chemical agonists may be specifically designed to mimic certain physiochemical properties of GDE. Antagonists may be any compound capable of blocking, inhibiting or otherwise preventing GDE from carrying out its normal biological functions. Antagonists include monoclonal antibodies specific for GDE or parts of GDE.

Analogues of GDE or of GDE agonistic or antagonistic agents contemplated herein include, for example, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

High Throughput Screening Assays

High throughput screening (HTS) typically uses automated assays to search through large numbers of compounds for a desired activity. Typically HTS assays are used to find new drugs by screening for chemicals that act on a particular receptor or molecule. For example, if a chemical inactivates an receptor it might prove to be effective in preventing a process in a cell which causes a disease. High throughput methods enable researchers to try out thousands of different chemicals against each target very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day. Examples include the yeast two-hybrid system and phage display. For examples of phage display see, U.S. Pat. Nos. 5,096,815; 5,198,346; 5,223,409; 5,260,203; 5,403,484; 5,534,621; and 5,571,698.

To achieve high-throughput screening, it is best to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known modulator (or activator) of an receptor for which modulators are sought, can be incubated with one sample of the assay, and the resulting decrease (or increase) in the receptor activity determined according to the methods herein. It will be appreciated that modulators can also be combined with the receptor activators or modulators to find modulators which inhibit the receptor activation or repression that is otherwise caused by the presence of the known the receptor modulator. Similarly, when ligands to a sphingolipid target are sought, known ligands of the target can be present in control/calibration assay wells.

Measuring Binding Reactions During Screening Assays

Techniques for measuring the progression of binding reactions in multicontainer carriers are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., The Chemist's Companion: A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol. I, pp. 155-194, CRC Press (1981).

In spectrofluorometric methods, receptors are exposed to substrates that change their intrinsic fluorescence when processed by the target receptor. Typically, the substrate is nonfluorescent and converted to a fluorophore through one or more reactions. As a non-limiting example, SMase activity can be detected using the Amplex.RTM. Red reagent (Molecular Probes, Eugene, Oreg.). In order to measure sphingomyelinase activity using Amplex Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (e.g., a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., Application of Fluorescence Polarization Assays in High-Throughput Screening, Genetic Engineering News, 17:27, 1997.

FP is particularly desirable since its readout is independent of the emission intensity (Checovich, W. J., et al., Nature 375:254-256, 1995; Dandliker, W. B., et al., Methods in Enzymology 74:3-28, 1981) and is thus insensitive to the presence of colored compounds that quench fluorescence emission. Fluoroecence Polarization (FP) and FRET (see below) are well-suited for identifying compounds that block interactions between sphingolipid receptors and their ligands. See, for example, Parker et al., Development of high throughput screening assays using fluorescence polarization: nuclear receptor-ligand-binding and kinase/phosphatase assays, J Biomol Screen 5:77-88, 2000.

Fluorophores derived from sphingolipids that may be used in FP assays are commercially available. For example, Molecular Probes (Eugune, Oreg.) currently sells sphingomyelin and one ceramide fluorophores. These are, respectively, N-(4,4-difluoro-5,7-dimethyl-4-bora-3a,4a-diaza-s-inda-cene-3-pentanoyl)sphingosyl phosphocholine (BODIPY.RTM. FL C5-sphingomyelin); N-(4,4-difluoro-5,7-dimethyl-4-bora-3a,4a-diaza-s-inda-cene-3-dodecanoyl)sphingosyl phosphocholine (BODIPY.RTM. FL C12-sphingomyelin); and N-(4,4-difluoro-5,7-dimethyl-4-bora-3a,4a-diaza-s-1-indacene-3-pentanoyl)sphingosine (BODIPY.RTM. FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION fluorescence polarization system (Tecan A G, Hombrechtikon, Switzerland). General multiwell plate readers for other assays are available, such as the VERSAMAX reader and the SPECTRAMAX multiwell plate spectrophotometer (both from Molecular Devices).

Fluorescence resonance energy transfer (FRET) is another useful assay for detecting interaction and has been described previously. See, e.g., Heim et al., Curr. Biol. 6:178-182, 1-996; Mitra et al., Gene 173:13-17 1996; and Selvin et al., Meth. Enzymol. 246:300-345, 1995. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res. 38:2365-2373 (1997); Kahl et al., Anal. Biochem. 243:282-283 (1996); Undenfriend et al., Anal. Biochem. 161:494-500 (1987)). See also U.S. Pat. Nos. 4,626,513 and 4,568,649, and European Patent No. 0,154,734. One commercially available system uses FLASHPLATE scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells.

The assay can be measured immediately or allowed to reach equilibrium. Either way, when a radiolabel becomes sufficiently close to the scintillant coating, it produces a signal detectable by a device such as a TOPCOUNT NXT microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., Anal. Biochem. 257:112-119, 1998).

In one embodiment, GDE protein family members are detected/quantified using a ligand binding assay, such as, for example, a radioligand binding assay. Briefly, a sample from a tissue expressing GDE protein family members is incubated with a suitable ligand under conditions designed to provide a saturating concentration of ligand over the incubation period. After ligand treatment, the sample is assayed for radioligand binding. Any ligand that binds to GDE protein family members can be employed in the assay. Any of the GDE protein family member modulators discussed above can, for example, be labeled and used in this assay. An exemplary, preferred ligand for this purpose is ¹²⁵I-omega-conotoxin GVIA. Binding of this ligand to cells can be assayed as described, for example, in Solem et al. (1997) J. Pharmacol. Exp. Ther. 282:1487-95. Binding to membranes (e.g., brain membranes) can be assayed according to the method of Wagner et al. (1995) J. Neurosci. 8:3354-3359 (see also, the modifications of this method described in McMahon et al. (2000) Mol. Pharm. 57:53-58).

Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence GDE polypeptide(s) in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In certain embodiments, GDE polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads TM), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., 3H, 125I, 35S, 14C, or 32P), receptors (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, late; etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In preferred embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Changes in GDE protein family member subunit expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In one embodiment, amplification-based assays can be used to detect, and optionally quantify, a polynucleotide encoding a GDE protein of interest. In such amplification-based assays, the mRNA in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR(RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of the GDE mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of GDE mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the GDE mRNA of interest, and standard methods (such as, e.g., densitometry where the nucleic acid probe is radioactively labeled) can be used to quantify the level of the GDE polynucleotide.). A variety of additional nucleic acid hybridization formats are known to those skilled in the art. Standard formats include sandwich assays and competition or displacement assays. Sandwich assays are commercially useful hybridization assays for detecting or isolating polynucleotides.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). See also, for example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

Hybridization assays according to the invention can also be carried out using a MicroElectroMechanical System (MEMS), such as the Protiveris' multicantilever array.

GDE RNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

The invention also provides a screening method based on determining the effect, if any, of a test agent on the level of the depolarization-induced inward current mediated by GDE protein family members. Agents that reduce this current can potentially reduce one or more drug-related effects and/or behaviors. Conversely, agents that increase this current can potentially enhance such drug-related effects and/or behaviors.

The current can be measured using any available technique An indirect measurement of current can be carried out described by McMahon et al. (2000) Mol. Pharm. 57:53-58). In this method, cells are loaded with a dye that fluoresces in the presence of (such as fura-2 AM) prior to depolarization. Cells are generally also preincubated in the presence or absence of an GDE protein family member-specific modulator (e.g., 1 uM omega-conotoxin GVIA) to determine the extent of the current that is attributable to GDE protein family members. Cells are subsequently depolarized by incubation in a 50 mM KCl buffer in the continued presence or absence of the modulator. The resulting current can then be calculated based on fluorescence, as described by Solem et al. (1997) J. Pharmacol. Exp. Ther. 282:1487-95. Ruiz-Velasco and Ikeda (J. Neuroscience (2000) 20:2183-91 describe the direct measurement of currents using a whole-cell variant of the patch-claim technique, which can also be employed in the present invention.

Cells useful for screening based on current include any of those described above in connection with screening based levels of GDE protein family members or polypeptides or RNA or described below in the Examples.

In one embodiment, the test agent is contacted with the cell in the presence of the drug. The drug is generally one that produces one or more undesirable effects or behaviors, such as, for example, sedative-hypnotic and analgesic drugs. In particular embodiments, the drug is ethanol, a cannabinioid, or an opioid.

In a preferred embodiment, generally involving the screening of a large number of test agents, the screening method includes the recordation of any test agent selected in any of the above-described prescreening or screening methods in a database of agents that may modulate a drug-related effect or behavior. The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

### Test Agents Identified by Screening

When a test agent is found to modulate one or more GDE protein family members, or RNA. A preferred screening method of the invention further includes combining the test agent with a carrier, preferably pharmaceutically acceptable carrier, such as are described above. Generally, the concentration of test agent is sufficient to alter the level of GDE protein family members or RNA, or differentiation. This concentration will vary, depending on the particular test agent and specific application for which the composition is intended. As one skilled in the art appreciates, the considerations affecting the formulation of a test agent with a carrier are generally the same as described above with respect to methods of reducing a drug-related effect or behavior.

In a preferred embodiment, the test agent is administered to an animal to measure the ability of the selected test agent to modulate a drug-related effect or behavior in a subject, as described in greater detail below.

Preferred compositions for use in the therapeutic methods of the invention inhibit the GDE protein family member function by about 5% based on, for example, compound state analysis techniques or modulatory profiles described infra, more preferably about 7.5% or 10% inhibition or initiation of differentiation of the cell, and still more preferable, at least about 20%, 30%, 40%, 50%, 60%, 70%, 80%, 90% or 100% initiation or inhibition of differentiation.

### Compositions

Soluble polypeptides derived from GDE protein family member that retain the ability to initiate differentiation are useful. In addition, modification of such residues may permit the skilled artisan to tailor the binding specificities and/or affinity of polypeptides.

The GDE protein family members are of particular interest because they are of interest in the treatment, prevention, amelioration, reduction or alleviation of diseases.

The polypeptides may be prepared in various ways including, for example, molecular biological techniques, including proteolytic digestion of cells or cellular membrane preparations comprising the receptor (Bartfeld et al., Active acetylcholine receptor fragment obtained by tryptic digestion of acetylcholine receptor from Torpedo californica, Biochem Biophys Res Commun. 89:512-9, 1979; Borhani et al., Crystallization and X-ray diffraction studies of a soluble form of the human transferrin receptor, J. Mol. Biol. 218:685-9, 1991), recombinant DNA technologies (Marlovits et al., Recombinant soluble low-density lipoprotein receptor fragment inhibits common cold infection, J Mol. Recognit. 11:49-51, 1998; Huang et al., Expression of a human thyrotrophin receptor fragment in Escherichia coli and its interaction with the hormone and autoantibodies from subjects with Graves' disease, J Mol. Endocrinol. 8:137-44, 1992), or by in vitro synthesis of oligopeptides.
Peptidomimetics

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be obviated with peptidomimetics.

Candidate, lead and other polypeptides having a desired biological activity can be used in the development of peptidomimetics with biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Specific examples of peptidomimetics are set forth below. These examples are illustrative and not limiting in terms of the other or additional modifications.

Peptides with a Reduced Isostere Pseudopeptide Bond

Proteases act on peptide bonds. Substitution of peptide bonds by pseudopeptide bonds may confer resistance to proteolysis or otherwise make a compound less labile. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.
Peptides with a Retro-Inverso Pseudopeptide Bond

To confer resistance to proteolysis, peptide bonds may also be substituted by retro-inverso pseudopeptide bonds (Dalpozzo, et al. (1993), hit. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.
Peptoid Derivatives

Peptoid derivatives of polypeptides represent another form of modified polypeptides that retain the important structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.
Polypeptides

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique [Merrifield (1964), J. Am. Chem. Soc., 85: 2149; Stewart and Young (1984), Solid Phase polypeptide Synthesis, Pierce Chemical Company, Rockford, Ill.; Bodansky and Bodanszky (1984), The Practice of polypeptide Synthesis, Springer-Verlag, New York; Atherton and Sheppard (1989), Solid Phase polypeptide Synthesis: A Practical Approach, IRL Press, New York].

Alternatively, polypeptides of this invention may be prepared in recombinant systems using polynucleotide sequences encoding the polypeptides. For example, fusion proteins are typically prepared using recombinant DNA technology.
Polypeptide Derivatives

A “derivative” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide.

Mutant Polypeptides: A polypeptide having an amino acid sequence identical to that found in a protein prepared from a natural source is a “wildtype” polypeptide. Mutant oligopeptides can be prepared by chemical synthesis, including without limitation combinatorial synthesis.

Mutant polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare mutant polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

Chemically Modified Polypeptides: As contemplated by this invention, the term “polypeptide” includes those having one or more chemical modification relative to another polypeptide, i.e., chemically modified polypeptides. The polypeptide from which a chemically modified polypeptide is derived may be a wildtype protein, a mutant protein or a mutant polypeptide, or polypeptide fragments thereof, an antibody or other polypeptide ligand according to the invention including without limitation single-chain antibodies, bacterial proteins and polypeptide derivatives thereof, or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

Polypeptides with N-Terminal or C-Terminal Chemical Groups: An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al. (1993), Pharma. Res. 10: 1268-1273). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

Polypeptides with a Terminal D-Amino Acid: The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Polypeptides With Substitution of Natural Amino Acids By Unnatural Amino Acids: Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. (1993), cited above).

Post-Translational Chemical Modifications: Different host cells will contain different post-translational modification mechanisms that may provide particular types of post-translational modification of a fusion protein if the amino acid sequences required for such modifications is present in the fusion protein. A large number (.about.100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues. Protein kinases catalyze such reactions. Phosphorylation is often reversible due to the action of a protein phosphatase, an receptor that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMct). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274:22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the receptors (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Amu. Rev. Physiol. 50:333-344, 1988, and Bradbury et al. Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

Substitutions encompass amino acid alterations in which an amino acid is replaced with a different naturally-occurring or a non-conventional amino acid residue. Such substitutions may be classified as “conservative”, in which case an amino acid residue contained in a peptide is replaced with another naturally-occurring amino acid of similar character, for example Gly to Ala, Asp to Glu, Asn to Gln or Tip to Tyr. Possible alternative amino acids include serine or threonine, aspartate or glutamate or carboxyglutamate, proline or hydroxyproline, arginine or lysine, asparagine or histidine, histidine or asparagine, tyrosine or phenylalanine or tryptophan, aspartate or glutamate, isoleucine or leucine or valine.

It is to be understood that some non-conventional amino acids may also be suitable replacements for the naturally occurring amino acids. Substitutions encompassed by the present invention may also be “non-conservative”, in which an amino acid residue which is present in a polypeptide is substituted with an amino acid having different properties, such as naturally-occurring amino acid from a different group (e.g. substituting a charged or hydrophilic or hydrophobic amino acid with alanine), or alternatively, in which a naturally-occurring amino acid is substituted with a non-conventional amino acid. Additions encompass the addition of one or more naturally occurring or non-conventional amino acid residues. Deletions encompass the deletion of one or more amino acid residues.

One of skill in the art can identify other peptides and understands that homologues and orthologues of these molecules are useful in the compositions and methods of the instant invention. Moreover, variants of the peptides, are useful in the methods and compositions of the invention.

One of skill in the art will understand that molecules that share one or more functional activities with the molecules identified above, but have differences in amino acid or nucleic acid sequence would be useful in the compositions and methods of the invention. For example, in a preferred embodiment, a polypeptide or biologically active fragment thereof has at least about 60%, 70%, 80%, 90%, 95%, 96%, 97%, 98%, 99%, or 100% identity with the polypeptide set forth as SEQ ID NO:1-2, or a fragment or variant thereof.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences that one of skill in the art could use to make the molecules of the invention. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to 13245 nucleic acid molecules of the invention. BLAST protein searches can be performed with the XBLAST program, score=50, wordlength=3 to obtain amino acid sequences homologous to 13245 protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used.

Vectors

Another aspect of the invention pertains to vectors, preferably expression vectors, containing a nucleic acid molecule encoding the fusion molecules, or components thereof, of the invention as described above. As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., fusion molecules comprising a chemokine receptor ligand and a toxin moiety).

The recombinant expression vectors of the invention can be designed for expression of the polypeptides of the invention in prokaryotic or eukaryotic cells. For example, the polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and 17 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn 10-lac fusion promoter mediated by a coexpressed viral RNA polymerase (T7 gn1). This viral polymerase is supplied by host strains BL21(DE3) or HMS174(DE3) from a resident prophage harboring a 17 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari, et al., (1987) EMBO J. 6:229-234), pMFa (Kudjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the polypeptides can be expressed in insect cells using baculovirus expression vectors. Baculovirus vectors available for expression of proteins in cultured insect cells (e.g., Sf9 cells) include the pAc series (Smith et al. (1983) Mol. Cell. Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banedji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect of the invention pertains to host cells into which a nucleic acid molecule encoding a fusion polypeptide of the invention is introduced within a recombinant expression vector or a nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a fusion polypeptide of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including phosphate or chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the polypeptide of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the polypeptides of the invention. Accordingly, the invention further provides methods for producing polypeptides using the host cells of the invention. In one embodiment, the method comprises culturing the host cell of the invention (into which a recombinant expression vector encoding a polypeptide of the invention has been introduced) in a suitable medium such that a polypeptides of the invention is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which coding sequences have been introduced. Such host cells can then be used to create non-human transgenic animals in which exogenous sequences have been introduced into their genome or homologous recombinant animals in which endogenous sequences have been altered. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like.

Provided herein, according to one aspect, are vectors encoding one or more GDE proteins or fragments or variants thereof. For example, one vector may contain GenBank (NCBI) accession # AY910750 (SEQ. ID. NO.: 1), or a fragment or variant thereof, e.g., the GDPD domain of SEQ. ID. NO.: 1.

Provided herein, according to one aspect, are isolated cell that recombinantly expresses one or more peptides identified by GenBank (NCBI) accession # AY910750 (SEQ ID NO. 1), or fragments or variants thereof as well as the other GenBank sequences identified herein and fragments and variants thereof.

Methods of Making the Molecules of the Invention

As described above, molecules of the invention may be made recombinantly using the nucleic acid molecules, vectors, host cells and recombinant organisms described above.

Alternatively, the peptide can be made synthetically, or isolated from a natural source and linked to the carbohydrate recognition domain using methods and techniques well known to one of skill in the art.

Further, to increase the stability or half life of the fusion molecules of the invention, the polypeptides may be made, e.g., synthetically or recombinantly, to include one or more peptide analogs or mimmetics. Exemplary peptides can be synthesized to include D-isomers of the naturally occurring amino acid residues or amino acid analogs to increase the half life of the molecule when administered to a subject.

Pharmaceutical Compositions

The nucleic acid and polypeptide fusion molecules (also referred to herein as “active compounds”) of the invention can be incorporated into pharmaceutical compositions. Such compositions typically include the nucleic acid molecule or protein, and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions of the instant invention may also include one or more other active compounds. Alternatively, the pharmaceutical compositions of the invention may be administered with one or more other active compounds. Other active compounds that can be administered with the pharmaceutical compounds of the invention, or formulated into the pharmaceutical compositions of the invention, include, for example, anti-inflammatory compounds.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Preferred pharmaceutical compositions of the invention are those that allow for local delivery of the active ingredient, e.g., delivery directly to the location of a tumor. Although systemic administration is useful in certain embodiments, local administration is preferred in most embodiments.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, kit or dispenser together with instructions, e.g., written instructions, for administration, particularly such instructions for use of the active agent to treat against a disorder or disease as disclosed herein, including a GDE related disorder. The container, pack, kit or dispenser may also contain, for example, a nucleic acid sequence encoding a peptide, or a peptide expressing cell. For research and therapeutic applications, an GDE protein family member modulator is generally formulated to deliver modulator to a target site in an amount sufficient to inhibit GDE protein family members at that site.

Modulator compositions or peptides of the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween TM, Pluronics TM, and PEG). In one embodiment, the pharmaceutically acceptable carrier is an aqueous pH-buffered solution.

Certain embodiments include sustained-release pharmaceutical compositions. An exemplary sustained-release composition has a semipermeable matrix of a solid hydrophobic polymer to which the modulator is attached or in which the modulator is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Where the modulator is a polypeptide, exemplary sustained release compositions include the polypeptide attached, typically via epsilon-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped modulator. Liposomes are small vesicles composed of various types of lipids, phospholipids, and/or surfactants. These components are typically arranged in a bilayer formation, similar to the lipid arrangement of biological membranes. Liposomes containing GDE protein family member modulators are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions can also include an modulator adsorbed onto a membrane, such as a silastic membrane, which can be implanted, as described in International Publication No. WO 91/04014.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide encoding a polypeptide modulator of GDE protein family members. Such compositions optionally include other components, as for example, a storage solution, such as a suitable buffer, e.g., a physiological buffer. In a preferred embodiment, the composition is a pharmaceutical composition and the other component is a pharmaceutically acceptable carrier as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)pro-pyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above.

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

Transgenic Animals

The transgenic non-human animal may be a primate, mouse, dog, cat, sheep, horse, rabbit or other non-human animal. Cells may be isolated and cultured from the transgenic non-human animals. The cells may be used in, for example, primary cultures or established cultures. In one aspect, provided herein are uses of a transgenic animal as described herein to test therapeutic agents.

In another embodiment, a decrease differentiation indicates that the test agent may be useful in treating a GDE disorder or changes in GDPD enzymatic activity.

A transgenic non-human animal comprising an over-expressed NTB peptide or a fragment or variant thereof. The use of a transgenic animal according to claim 50, to test therapeutic agents. Embodiments of the invention include the use of the ES cell lines derived from the transgenic zygote, embryo, blastocyst or non-human animal to treat human and non-human animal diseases.

Transgenic non-human animals include those whose genome comprises over-expressed NT_{B} peptide or a fragment or variant thereof comprising the nucleic acid sequence set forth in SEQ ID NO: 1-3, or a fragments or variants thereof. The methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, Calif., Ed., Transgenic Animal Technology: A Laboratory Handbook, Academic Press, 1994. In certain embodiments, transgenic mice will be produced as described in Thomas et al. (1999) Immunol., 163:978-84; Kanakaraj et al. (1998) J. Exp. Med., 187:2073-9; or Yeh et al. (1997) Immunity 7:715-725. Methods of producing the transgenic animals are well-known in the art. See for example, Hooper, M L, Embryonal Stem Cells: Introducing Planned Changes into the Animal Germline (Modeem Genetics, v. 1), Int'. Pub. Distrib., Inc., 1993; Bradley et al. (1984) Nature, 309, 255-258; Jaenisch (1988) Science, 240:1468-1474; Wilmut et al. (1997) Nature, 385: 810-813; DeBoer et al., WO 91/08216; Wang, et al. Molecular Reproduction and Development (2002) 63:437-443); Page, et al. Transgenic Res (1995) 4(6):353-360; Lebkowski, et al. Mol Cell Biol (1988) 8(10):3988-3996; “Molecular Cloning: A Laboratory Manual. Second Edition” by Sambrook, et al. Cold Spring Harbor Laboratory: 1989; “Transgenic Animal Technology: A Laboratory Handbook,” C. A. Pinkert, editor, Academic Press, 2002, 2nd edition, 618 pp.; “Mouse Genetics and Transgenics: A Practical Approach,” I. J. Jackson and C. M. Abbott, editors, Oxford University Press, 2000, 299 pp.; “Transgenesis Techniques: Principles and Protocols,” A. R. Clarke, editor, Humana Press, 2001, 351 pp.; Velander et al., Proc. Natl. Acad. Sci. USA 89:12003-12007, 1992; Hammer et al., Nature 315:680-683, 1985; Gordon et al., Science 214:1244-1246, 1981; and Hogan et al., Manipulating the Mouse Embryo: A Laboratory Manual (Cold Spring Harbor Laboratory, 2002), which are each incorporated herein by reference in their entirety.

Cells obtained from the transgenic non-human animals described herein may be obtained by taking a sample of a tissue of the animal. The cells may then be cultured. The cells preferably lick production of functional protein encoded by the nucleotide sequence comprising SEQ ID NO: 1-3 or a fragments or variants thereof.

In one embodiment, the transgenic non-human animal is a male non-human animal. In other preferred embodiments the transgenic non-human animal is a female non-human animal. According to other embodiments, the transgenic non-human animal oocyte, blastocyst, embryo, or offspring may be used as a model for a human disease, as a model to study human disease or to screen molecules, compounds and compositions. In certain embodiments, the cells of the transgenic oocyte, zygote, blastocyst, or embryo are used to establish embryonic stem (ES) cell lines. Stem cells are defined as cells that have extensive proliferation potential, differentiate into several cell lineages, and repopulate tissues upon transplantation. (Thomson, J. et al. 1995; Thomson, J. A. et al. 1998; Shamblott, M. et al. 1998; Williams, R. L. et al. 1988; Orkin, S. 1998; Reubinoff, B. E., et al. 2000).

Kits

The invention also provides kits useful in practicing the methods of the invention. In one embodiment, a kit of the invention includes a GDE protein family member modulator, e.g., contained in a suitable container. Provided herein, according to one aspect, are kits comprising an GDE modulator and a pharmaceutically acceptable carrier and b) instructions for use. In a variation of this embodiment, the GDE protein family member modulator is formulated in a pharmaceutically acceptable carrier. The kit preferably includes instructions for administering the N-type modulator to a subject to reduce or prevent a drug-related effect or behavior.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Example 1

Differential Subtraction Screen

Ventral neural explants were dissected from brachial level HH St17 chick embryos and cultured as described previously for 18 hours (1). The screen was designed to maximize isolation of genes responsive to motor neuron-derived retinoids however genes responsive to paraxial mesoderm-derived retinoids would also have been included. RNA from 100 explants was isolated using Trizol (GIBCO-BRL). cDNAs were generated and amplified using the Marathon cDNA PCR Amplification kit (Stratagene) and the subtraction procedure carried out using the Stratagene PCR Select Suppression kit. Reverse Northern blot analysis was performed as described in the latter kit.

Cloning

A full-length cDNA clone of GDE2 was obtained using standard cDNA library screens and 5′Rapid Amplification of cDNA Ends (RACE). This corresponded to a single transcript of 4.2 kb detected by Northern analysis.

In Situ Hybridization and Immunofluorescent Staining

In situ hybridization and immunostaining were performed as previously described (2). The GDE2 antibody is an affinity-purified rabbit polyclonal antibody raised against a 14 amino acid C-terminal peptide, and used at 1:20,000. Dilutions of remaining antibodies (1, 3) are as follows: guinea pig anti-Nkx6.1 1:4000; guinea pig anti-Olig2 1:20,000; mouse anti-p27^{kip1} 1:50 (BD Laboratories); mouse anti-MNR21HB9 1:100 (DSHB); rabbit anti-HB9 1:2000; guinea pig anti-Islet1/2 1:10,000; mouse anti-Islet2 1:1.00 (DSHB); goat anti-β-Gal 1:3000 (Arnel); mouse anti-Pax6 1:30 (DSHB); mouse anti-Nlac2.2 1:100 (DSHB); mouse anti-FLAG 1:200 (Stratagene); rat anti-BrdU 1:100 (Sigma). For BrdU labeling, the vitelline membrane was removed, 100 μM BrdU/PBS was applied directly to the embryo, embryos were incubated at 39° C. for 30 min, and then processed for immunostaining. Confocal micrographs were captured on a Zeiss LSM 5 PASCAL microscope.

In Vivo Analysis in Chick Embryos

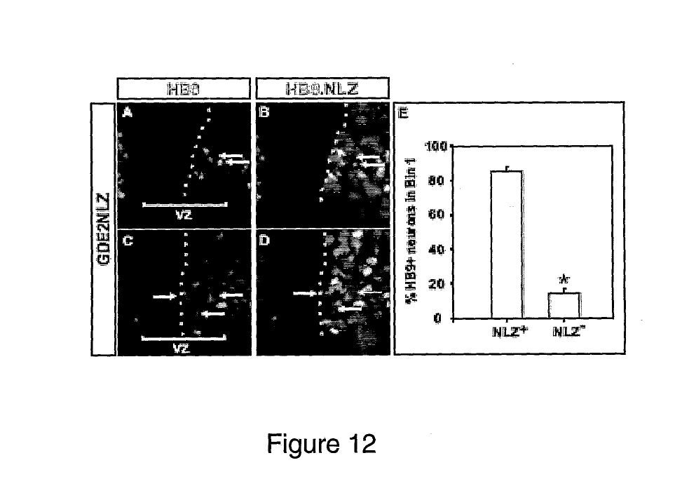

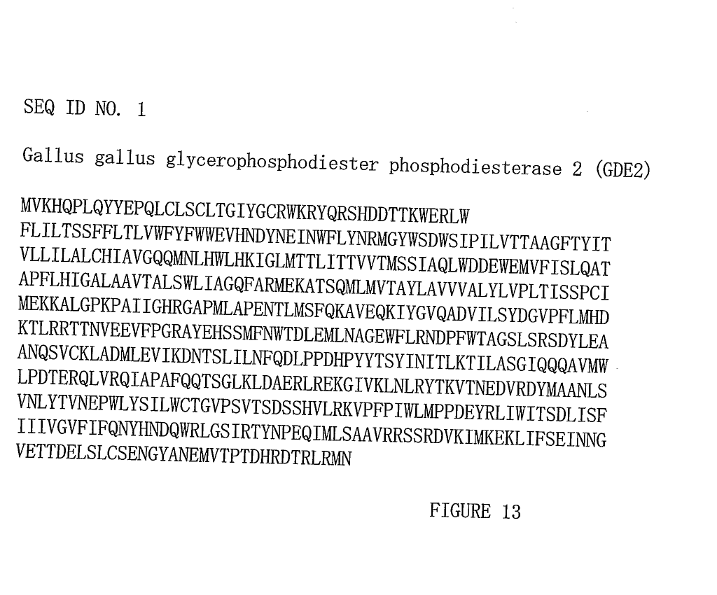

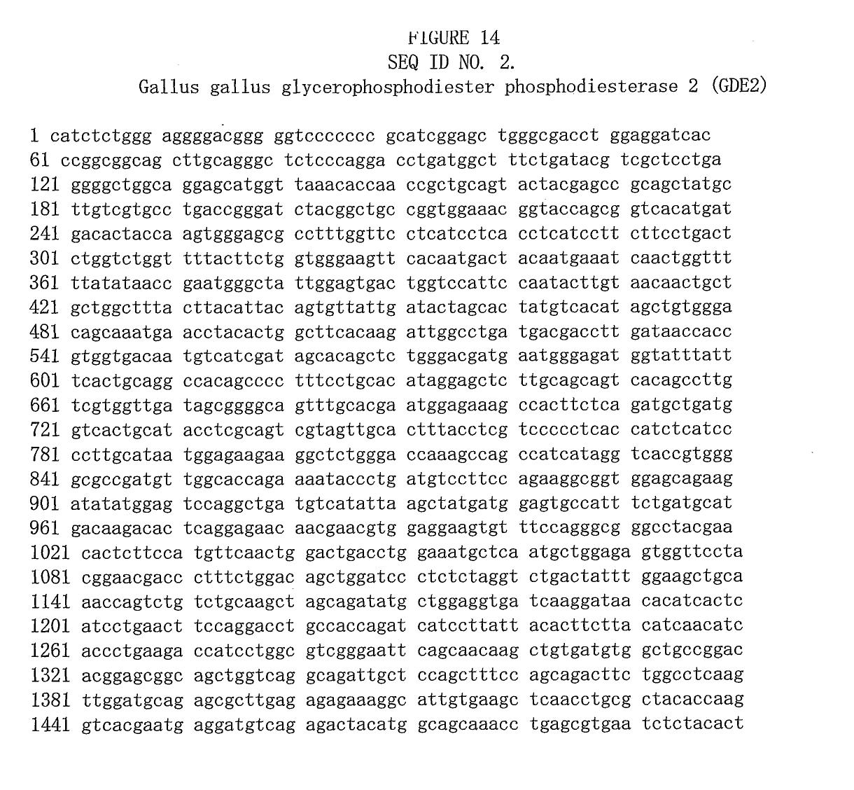

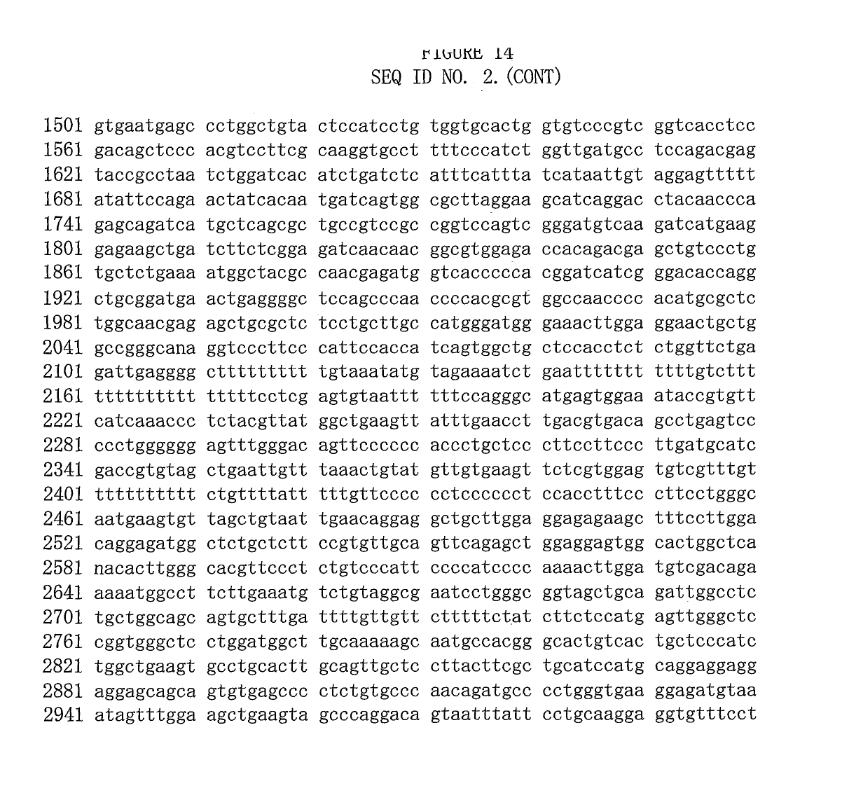

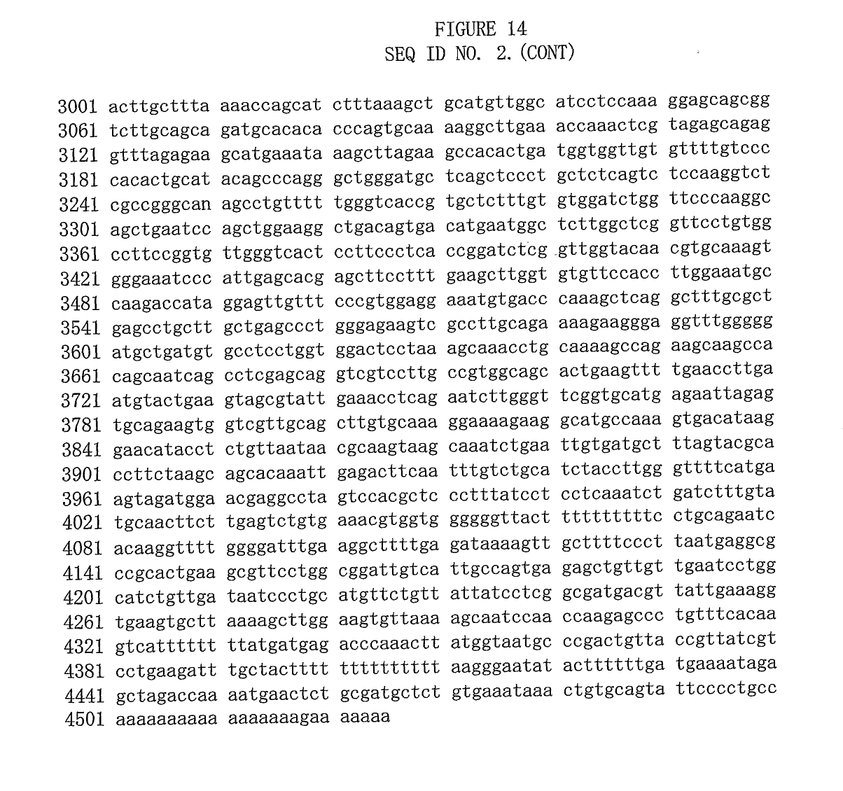

For loss-of-function experiments, embryos were electroporated with siRNAs as previously described (2) and analyzed 40 hours later at HH St19-21. GDE2 siRNA target sequences are as follows: 5′-AAUCCAGCUGGAAGGCUGACA-3′, and 5′-AAAGCUCAGGCUUUGCGCUGA-3′. DsRed siRNA sequences have been previously published (2). For quantitation, 5-7 sections from each of 5 embryos with at least 70% loss of GDE2 expression were scored for marker expression. For gain-of-function experiments, embryos were electroporated at HH St13-14 with either GDE2NLZ or a NLZ control plasmid and then analyzed 24h later at HH St13-20. Images of 5-8 sections were captured from each of 5 embryos, the electroporated half of the spinal cord was divided into 3 bins, and marker expression was scored. For quantitative analysis of non cell-autonomy (FIG. 12), coincidence of GDE2 and NLZ expression was analyzed both by in vitro transfection as well as in ovo electroporation to confirm that cells which do not stain for NLZ do not express GDE2. In addition, during confocal imaging the gain for NLZ detection was significantly increased to ensure that all NLZ-stained cells were detected. The GDE2H.ANLZ construct was generated by site-directed mutagenesis using the Quikchange kit (Stratagene).

Cell Culture

HEK293 cells were cultured in DMEM+10% FBS and transfected using Lipofectamine 2000 (Invitrogen). Cells were fixed 24h after transfection for 10 min in 4% PFAIO1M PB. When noted, cells were permeabilized for 10 min in PBS+0.1% Triton-X. Nuclei were visualized by staining with TO-PRO3 at 1:10,000 (Molecular Probes).
1. S. Sockanathan, T. M. Jessell, Cell 94, 503 (1998).
2. M. Rao, J. H. Baraban, F. Rajaii, S. Sockanathan, Dev. Dyn. 231, 592 (2004).
3. B. G. Novitch, A. I. Chen, T. M. Jessell, Neuron 31, 773 (2001).

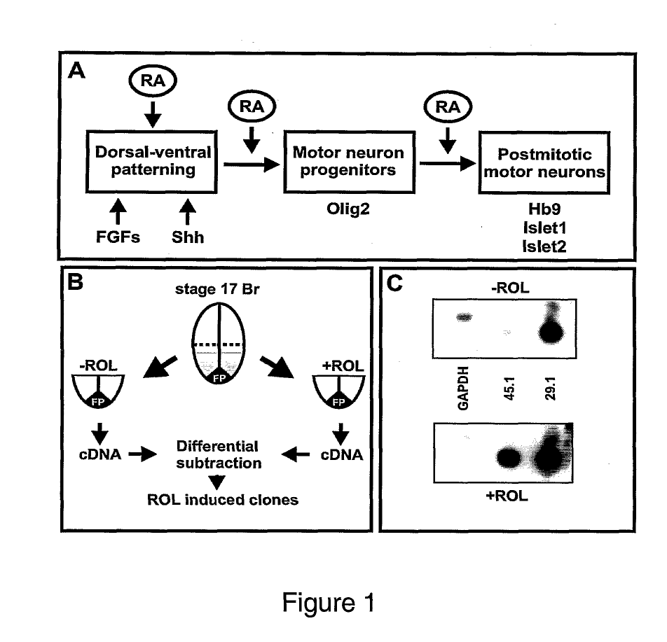

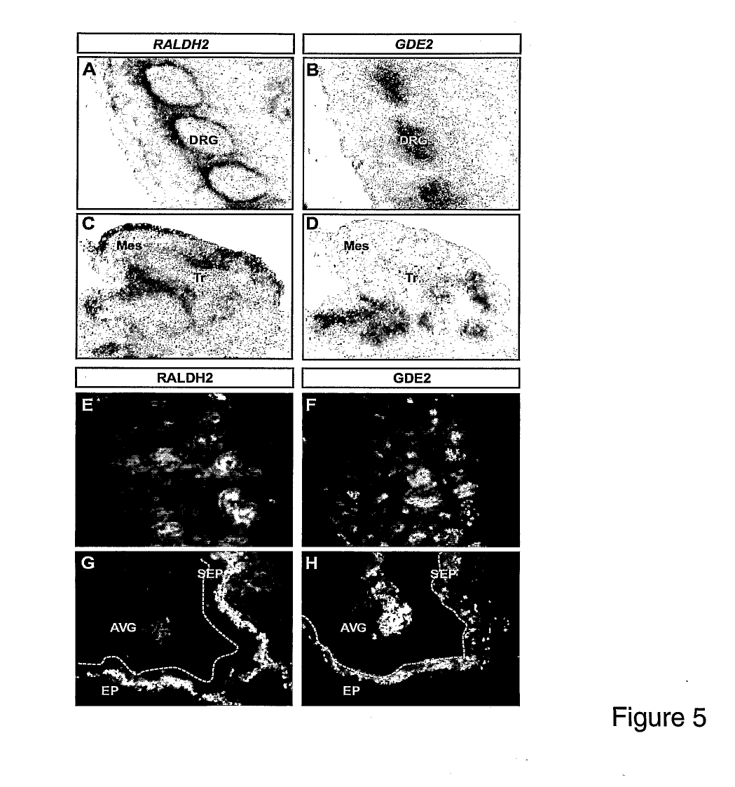

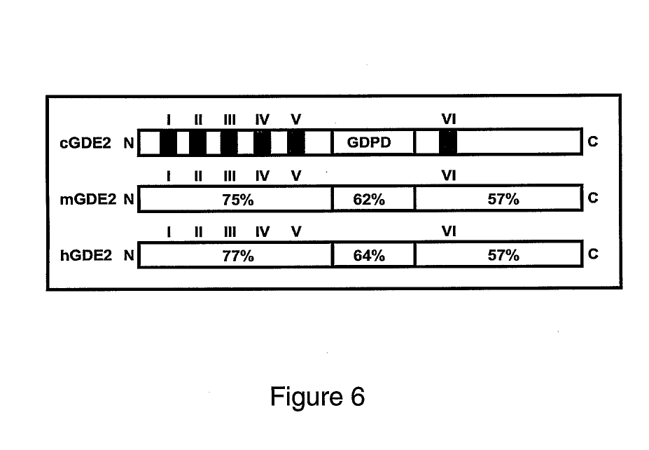

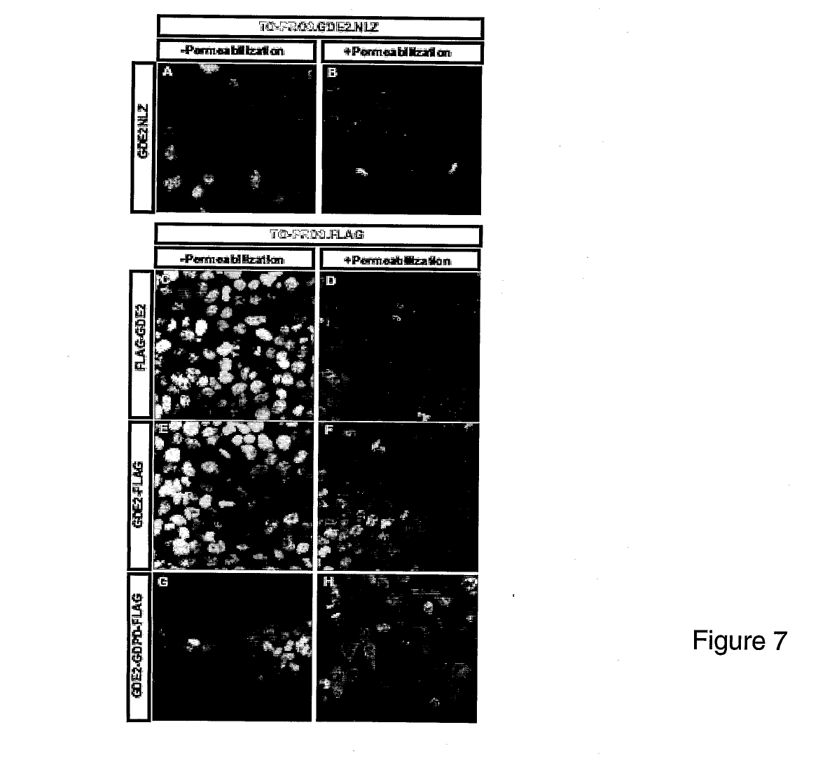

Spinal motor neuron generation in the chick requires the integration of three different extrinsic signals: sonic hedgehog, fibroblast growth factors, and retinoic acid (RA) (2, 3). All three signaling pathways have been implicated in initial dorsal-ventral patterning of progenitor domains in the spinal cord (FIG. 1A). However, RA signaling is also necessary for the induction of Oligodendrocyte transcription factor 2 (Olig2) in progenitors, and their subsequent differentiation into postmitotic motor neurons (FIG. 1A) (2). When motor neuron progenitors differentiate, they decrease expression of Olig2 as they migrate out of the ventricular zone (VZ) and increase expression of postmitotic motor neuron markers such as Islet1 and Islet2 (FIG. 1A) (4). Olig2 has a pivotal role in motor neuron differentiation. It is required for the maintenance of a motor neuron progenitor state and its downregulation is essential for the implementation of neurogenic and motor neuron specification pathways (5, 6). Because the differentiation of motor neuron progenitors is dependent upon retinoid signaling, a differential subtraction screen with cDNAs derived from ventral spinal cord explants grown in the presence or absence of retinol to identify genes involved in this process was conducted (FIG. 1B) (7). Probing reverse Northern blots with cDNAs from both sets of explants demonstrated that expression of clone 45.1 was increased approximately 50-fold in explants exposed to retinol, when compared with that of glyceraldehyde 3-phosphate dehydrogenase (GAPDH)(FIG. 1C). Furthermore, in situ hybridization analysis revealed that clone 45.1 was expressed within or directly adjacent to developing tissues that synthesize RA such as the spinal cord, paraxial mesoderm, mesonephros, heart, lung and eye (FIG. 5, 8). Sequence analysis revealed that clone 45.1 is a chick gene (AY910750) encoding a predicted protein of 599 amino acids with 67% identity to the human predicted protein PP 1665 and 66% identity to mouse Glycerophosphodiester Phosphodiesterase 2 (GDE2) (9, 10) (FIG. 6), suggesting clone 45.1 is the chick homologue of GDE2. These proteins all contain a glycerophosphodiester phosphodiesterase (GDPD) domain, known to be involved in glycerophosphodiester metabolism (11). Analysis of the Conserved Domain Database revealed that GDE2 is a member of a large, heterogeneous family of GDPD-containing proteins for which in vivo functions are largely unknown (9). GDE2 is a transmembrane protein and epitope tagging studies demonstrated that the GDPD domain is extracellular with intracellular localization of the N- and C-termini (FIG. 7).

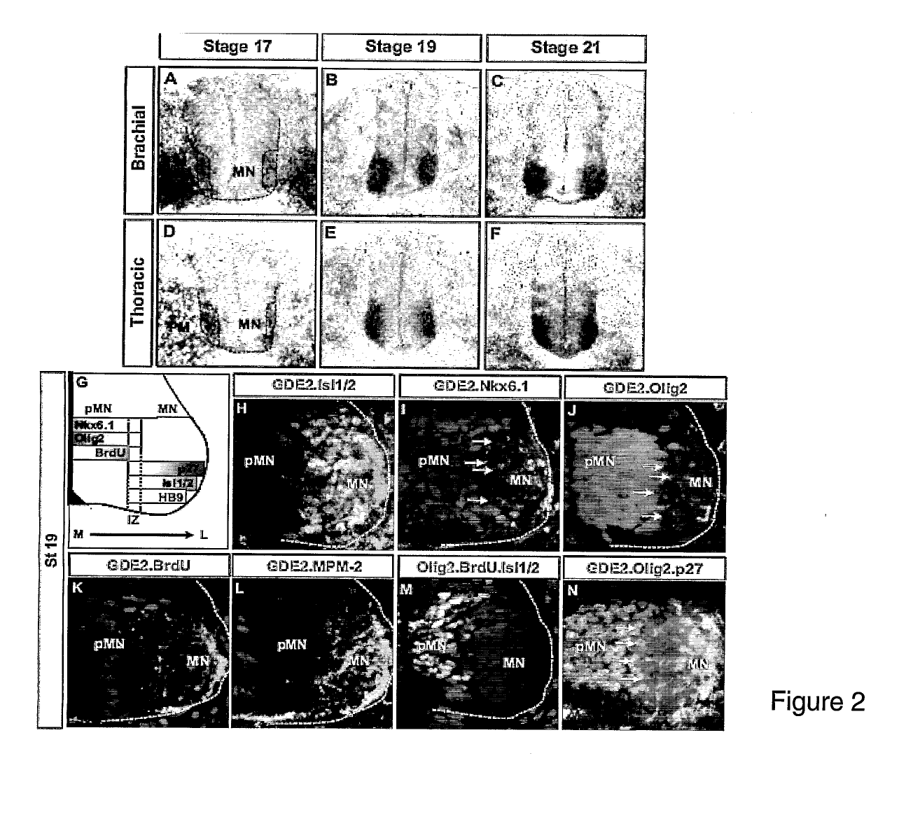

GDE2 is highly expressed by all somatic spinal motor neurons irrespective of their rostrocaudal position from the time they are generated (FIG. 2A-F) until at least Hamburger-Hamilton (HH) St29 (8). These data are consistent with the induction of GDE2 expression by paraxial mesoderm-derived RA signaling. In order to determine when GDE2 might act in motor neuron development, the onset of GDE2 expression was examined. The differentiation of motor neuron progenitors can be monitored accurately by the sequential expression of molecular markers as well as the position of their cell-bodies along the medial-lateral axis of the spinal cord. Actively cycling motor neuron progenitors located in the VZ of the spinal cord express large amounts of the transcription factor NK-homeobox 6.1 (Nkx6.1) and Olig2 (4, 6) (FIG. 2G). These progenitor markers are extinguished as the cells exit the cell-cycle, migrate laterally, and begin to express motor neuron-specific transcription factors such as Homeobox factor 9 (HB9), Islet1, and Islet1 (4) (FIG. 2G). GDE2 was localized in postmitotic, laterally-located neurons that also expressed HB9, Islet1, and Islet2 (FIG. 2H, 8), but was not detected in medially-located progenitor cells that expressed Nkx6.1 and Olig2 (FIG. 2, I and J). However, an intermediate population of cells weakly-stained for Nkx6.1 and Olig2 also contained GDE2 suggesting that GDE2 expression may be initiated as cells transition to a postmitotic state (arrows, FIG. 2, I and J).

Once ventral neuronal progenitors undergo their terminal mitosis at the medial margin of the VZ, resulting daughter cells migrate laterally into the intermediate zone (IZ) (12). In the IZ, they increase expression of the cyclin-dependent kinase inhibitor p27 (13), undergo cell-cycle arrest, and respond to signals that trigger terminal differentiation (FIG. 2G). In embryos incubated with bromodeoxyuridine (BrdU), GDE2 was not detected in any cells that incorporated BrdU or were stained by the antibody MPM-2 (14), indicating that GDE2 is not expressed by progenitors undergoing S- or M-phase in the VZ (FIG. 2, K and L). The border between the VZ and the IZ is defined by S-phase nuclei labeled by BrdU (12). Lateral to this border, there was a subset of BrdU-labeled cells that expressed Olig2 as well as GDE2 (FIG. 2, K and M). Consistent with their location in the IZ, these cells contained small amounts of the cell-cycle inhibitor p27 (FIG. 2N). In summary, GDE2 was primarily expressed by mature motor neurons however its expression was initiated within cells in the IZ as they differentiated into postmitotic motor neurons.

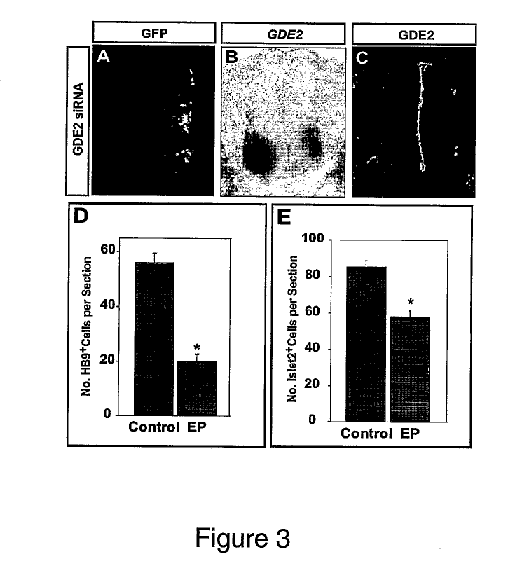

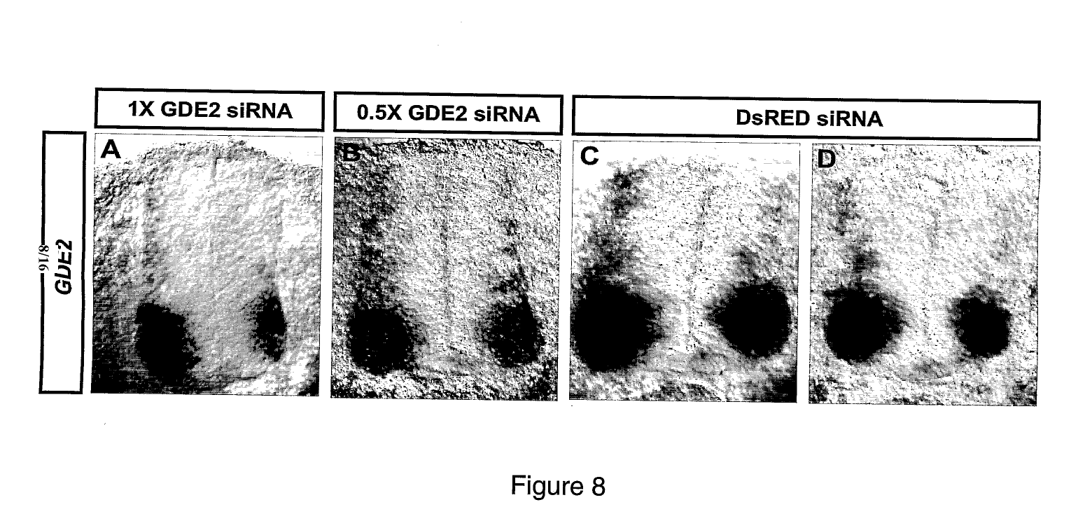

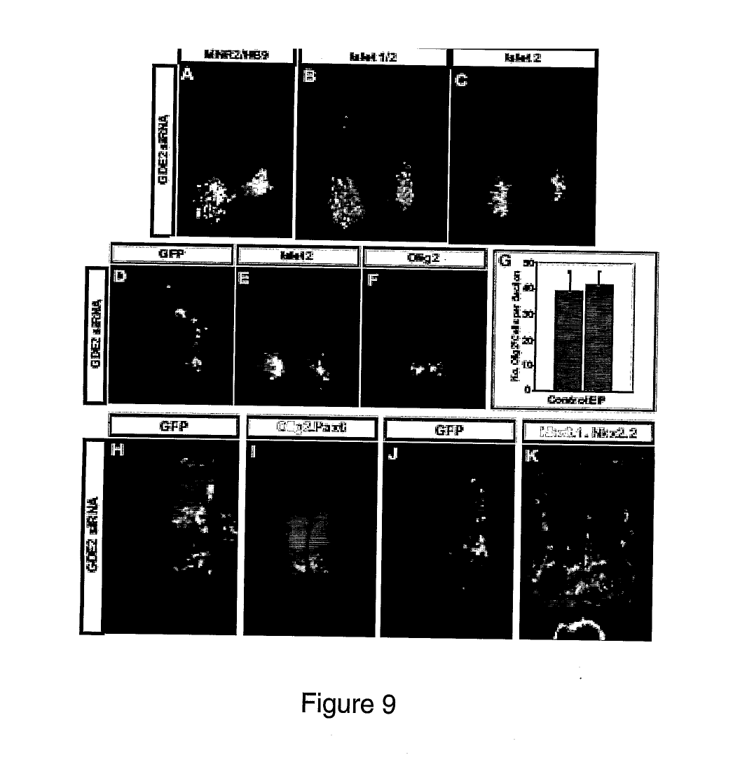

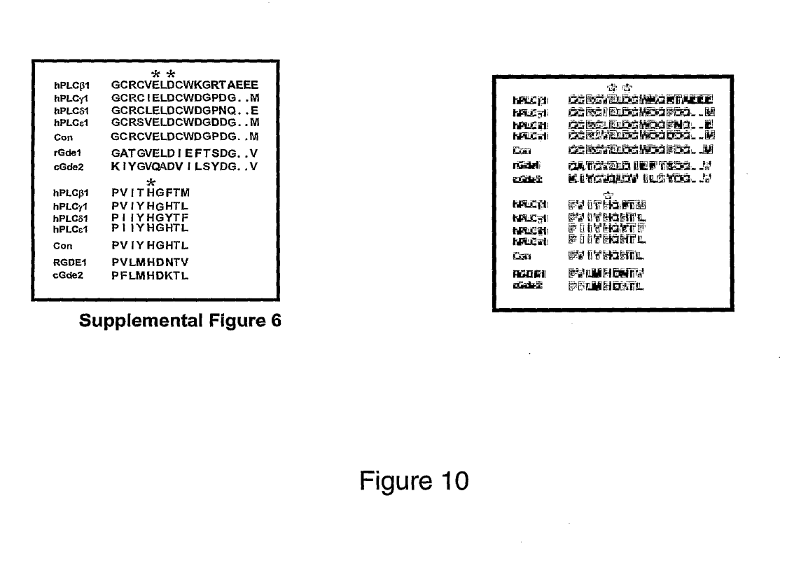

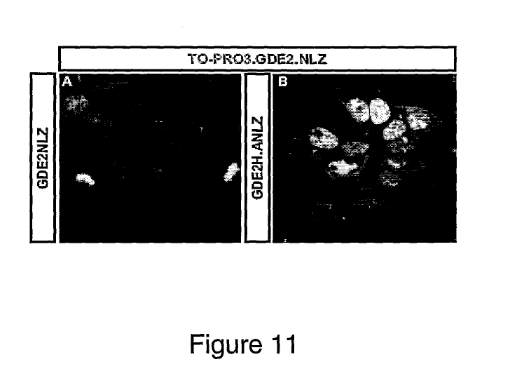

To test whether GDE2 might mediate the retinoid-dependent differentiation of Olig2 progenitors, we ablated GDE2 expression in the spinal cord by in ovo electroporation of small interfering RNAs (siRNA) (15). The experiments used a green fluorescent protein (GFP) reporter plasmid to identify the electroporated side of the spinal cord. Electroporation of GDE2 siRNA typically resulted in a 70% loss of GDE2 mRNA and protein in spinal motor neurons (FIG. 3, A through C). Loss of GDE2 expression depended on the amount of siRNA administered, and siRNAs directed against different parts of the GDE2 open reading frame and 3′ untranslated region resulted in a similar loss of GDE2 mRNA and protein (FIG. 8, 8). GDE2 silencing was not triggered by unrelated siRNAs, and GDE2 siRNAs did not induce global changes in gene expression (FIGS. 8 and 9). No toxicity was detected by terminal deoxynucleotidyl transferase biotin-dUTP nick end labeling (TUNEL) (15).

Embryos lacking GDE2 were analyzed for expression of the postmitotic motor neuron markers HB9, Islet1, and Islet2 by immunohistochemistry on the same or serial sections. In all cases a marked decrease in the number of neurons expressing each of these markers was evident on the electroporated side of the spinal cord, with approximately 70% loss of HB9-expressing neurons and 30-40% loss of more mature motor neurons expressing Islet2 (FIG. 3, D and E; FIG. 9). Mice lacking HB9 show a progressive loss of Islet1-expressing cells while they maintain normal numbers of Islet2-expressing motor neurons suggesting that separate pathways of motor neuron differentiation may exist (16, 17). Our observation that GDE2 silencing impacts HB9 expression more severely than Islet2 indicates a differential requirement for GDE2 activity in these two pathways. We found no expansion in the number of neighboring interneurons but an increase in TUNEL together with a reduction in the width of the electroporated ventral spinal cord was observed (FIG. 3, A through C; 8). Without wishing to be bound by any particular scientific theories, this suggests that GDE2 silencing results in the loss of postmitotic motor neurons and that cells destined to become motor neurons likely do not convert to a different fate but instead undergo cell death.

To confirm that the loss of motor neurons upon GDE2 silencing did not result from defects in progenitor generation or proliferation, expression of the progenitor marker Olig2 and that of the ventral patterning genes Paired Box 6 (Pax6), Nkx2.2 and Nkx6.1 was analyzed in embryos electroporated with GDE2 siRNA. There was no change in the dorsal-ventral boundaries of the motor neuron progenitor domain or in the number of cells expressing Olig2 (FIG. 9). Consistent with this, there was also no change in the number of cells expressing Motor Neuron Restricted 2 (MNR2), a transcription factor turned on by committed progenitors in S-phase of the final cell-cycle and maintained throughout their differentiation (8, 14). Thus, GDE2 silencing appears not to affect progenitor cell generation or number.

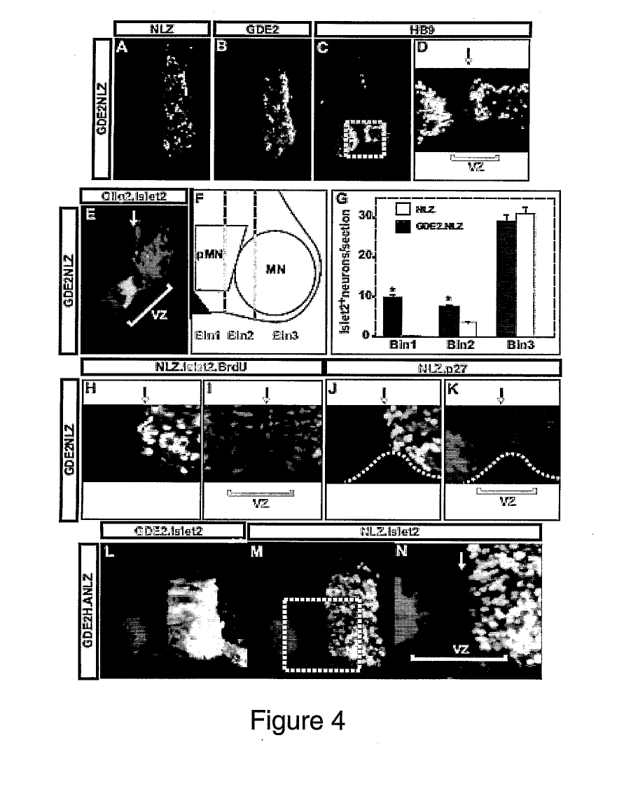

To test whether GDE2 is sufficient to drive motor neuron differentiation GDE2 was misexpressed throughout the spinal cord including within cycling Olig2-expressing progenitors in the VZ. A bicistronic construct was engineered with GDE2 linked to an internal ribosomal entry site (IRES) upstream of a nuclear form of β-galactosidase (GDE2NLZ) under the control of the chick β-actin promoter. Electroporation of GDE2NLZ into chick spinal cords resulted in high coincident expression of GDE2 and NLZ along the entire mediolateral axis in both progenitors and postmitotic neurons (FIG. 4, A and B). In contrast to the unelectroporated side, many medial cells in the electroporated VZ expressed the motor neuron marker HB9 (FIG. 4, C and D). Furthermore all of these medial HB9-containing cells also expressed markers of terminal motor neuron differentiation such as Islet2 (FIG. 4E) and choline acetyltransferase, the enzyme required for biosynthesis of the motor neuron neurotransmitter acetylcholine (8, 18). To quantify this effect, we divided the ventrolateral spinal cord into 3 bins which approximately corresponded to domains of motor neuron progenitors, differentiating motor neurons, and postmitotic motor neurons (FIG. 4F). More Islet2-expressing neurons were detected in Bin1 and Bin2 of embryos electroporated with GDE2NLZ than in embryos electroporated with NLZ alone (FIG. 4G). However, similar numbers of NLZ-expressing cells were detected in each case (FIG. 4).

Cells differentiating in response to GDE2 within the VZ expressed large amounts of the cell-cycle inhibitor p27, and failed to incorporate BrdU (FIG. 4, H through K). Moreover, these Islet2-expressing cells in the VZ had decreased expression of Sry-related HMG box 1 (Sox1) and Sox2, transcription factors required for maintenance of neural progenitor status (8, 19, 20). Finally, GDE2NLZ-electroporated embryos showed a corresponding loss of Olig2 within the VZ and no cells expressing both Olig2 and Islet2 were detected (FIG. 4E). However, the motor neurons generated in response to GDE2 misexpression were confined to the dorsal-ventral limits of the domain containing Olig2-expressing progenitors suggesting a prior requirement for Olig2 expression in these cells (FIG. 4E). Promoting cell-cycle exit in the developing spinal cord is not sufficient to elicit terminal differentiation of motor neurons (19, 21). Our results demonstrate that GDE2 is not only capable of driving cell-cycle exit, but can coordinately downregulate progenitor determinants and promote the differentiation of motor neuron progenitors into mature motor neurons.

The presence of the GDPD domain in GDE2 invokes the possibility that its atalytic activity may be required for its function. The related two-transmembrane protein GDE1 can hydrolyze glycerophosphoinositol (GPI), GPI-4,5-bisphosphate, and glycerophosphoserine and this activity is dependent on the integrity of the GDPD domain (9). The GDPD domain of GDE1 is 51% similar to the catalytic X-domain of phosphoinositide phospholipase C(PI-PLC)(22)(FIG. 10) and three amino acids essential for PI-PLC catalytic activity are conserved (23, 24). One of these three amino acids, a Histidine, is also crucial for GDE1-mediated hydrolysis of GPI (9). Because the location of this Histidine residue is conserved in the GDPD domain of GDE2 (FIG. 10), we altered it to Alanine (GDE2H.A) and determined whether the mutated protein could still promote ectopic motor neuron differentiation. Electroporation of GDE2H.ANLZ resulted in many electroporated cells within the VZ that expressed both NLZ and GDE2 (FIG. 4, L and M). However, no motor neurons expressing Islet2 were detected (FIG. 4N). Transfection of GDE2H.ANLZ into HEK293 cells revealed no difference in level of expression or membrane localization compared to transfection of GDE2NLZ (Figure S11). Thus a single amino acid change within the putative catalytic site of the GDPD domain in GDE2 is sufficient to abolish the ability of GDE2 to promote motor neuron differentiation, providing strong evidence that GDPD activity is required for GDE2 function.

The extracellular orientation of the GDPD domain raises the possibility that it may act non cell-autonomously. GDE2NLZ was electroporated into chick spinal cords and analyzed the number of ectopic motor neurons expressing NLZ. If GDE2 can function non cell-autonomously, a fraction of the }1B9-expressing neurons in Bin1 (FIG. 4F) should be untransfected and lack both NLZ and GDE2 expression. Although 85% of the HB9-containing cells in Bin 1 did express NLZ, 15% did not but were in direct contact with GDE2-expressing cells (FIG. 12). Thus, GDE2 function appears to be primarily cell-autonomous but may also be non cell-autonomous locally, at high concentrations. Consistent with this, spinal cord explants grown in media conditioned by GDE2-expressing cells do not exhibit premature motor neuron differentiation (8).

Paraxial mesoderm-derived RA may induce expression of GDE2 in cells poised to differentiate into postmitotic motor neurons. The GDPD activity of GDE2 is required for its ability to promote cell-cycle exit and motor neuron differentiation, and this may result directly from reducing amounts of Olig2 (5). The extracellular location of the GDPD domain distinguishes it from other known proteins involved in lipid signaling (22) but, the downstream pathways are unknown. Without wishing to be bound by any particular scientific theoris, one possibility is that GDE2 could act in concert with G-protein signaling pathways by analogy to GDE1 which interacts with members of the RGS (Regulators of G-protein signaling) family of proteins (25). A related protein GDE3 induces the differentiation of osteoblast-like cell lines in vitro (16) raising the possibility that six-transmembrane GDPD-containing proteins may constitute a family of critical cell differentiation factors.

### REFERENCES

1. C. Kintner, JNeurosci., 22, 639 (2002).
2. B. Novitch, H. Wichterle, T. M. Jessell, S. Sockanathan, Neuron 40, 81 (2003).
3. R. Diez Del Corral, et al., Neuron 40, 65 (2003).
4. R. Shirasaki, S. L. Pfaff, Annu. Rev. Neurosci., 25, 251 (2002).
5. S-K. Lee, B. Lee, E. C. Ruiz, S. L. Pfaff, Genes and Dev. 19, 282 (2005).
6. B. G. Novitch, A. I. Chen, T. M. Jessell, Neuron 31, 773 (2001).
7. Materials and methods are available as supporting material on Science online.
8. M. Rao and S. Sockanathan, unpublished data.
9. B. Zheng, C. P. Berrie, D. Corda, M. G. Farquhar, Proc. Natl. Acad. Sci. U.S.A. 100, 1745 (2003).
10. Y. Nogusa, Y. Fujioka, R. Komatsu, N. Kato, N. Yanaka, Gene 337, 173 (2004).
11. J. Tommassen, et al., Mol. Gen. Genet. 220, 321 (1991).
12. M. Hollyday, Int. J. Dev. Neurosci. 19, 161 (2001).
13. M. H. Farah, et al., Development 127, 693 (2000).
14. Y. Tanabe, C. William, T. M. Jessell Cell 95, 67 (1998).
15. M. Rao, J. H. Baraban, F. Rajaii, S. Sockanathan, Dev. Dyn. 231, 592 (2004).
16. S. Arber, et al., Neuron 23, 659 (1999).
17. J. Thaler, et al., Neuron 23, 675 (1999).
18. P. E. Phelps, R. P. Barber, J. E. Vaughn, J. Comp Neurol. 307, 77 (1991).
19. V. Graham, J. Khudyakov, P. Ellis, L. Pevny. Neuron 39, 749 (2003).
20. M. Bylund, E. Andersson, B. G. Novitch, J. Muhr. Nat. Neurosci. 6, 1162 (2003).
21. R. Mizuguchi, et al., Neuron 31, 757 (2001).
22. M. J. Rebecchi, S. N. Pentyala, Physiol. Rev. 80, 1291 (2000).
23. L-O. Essen, et al., Biochem. 36, 1704 (1997).
24. H-F. Cheng, et al., J. Biol. Chem. 270, 5495 (1995).
25. B. Zheng, D. Chen, M. G. Farquhar, Proc. Natl. Acad. Sci. U.S.A. 97, 3999 (2000).
26. N. Yanaka, et al., l Biol. Chem. 44, 43595 (2003).

1. A method of modulating cellular differentiation, comprising modulating the functional level of a glycerophosphodiester phosphodiesterase (GDE) protein wherein inducing over-expression of the GDE protein level or decreasing functional levels of GDE protein modulates differentiation of a cell.

2. A method of modulating cellular differentiation in a mammal, comprising modulating the functional level of a GDE proteins wherein inducing over-expression of the GDE protein level or decreasing functional levels of GDE protein modulates differentiation of the a cell.

3. A method for the treatment and/or prophylaxis of a condition characterized by aberrant or otherwise unwanted cellular differentiation in a mammal, comprising modulating the functional level of a GDE protein in the mammal, wherein inducing over-expression of the GDE protein level or decreasing functional levels of GDE protein modulates differentiation of the cells.

4. The method according to claim 1, wherein the cell is one or more of a neuronal cell, a pancreatic cell, a lung cell, bone tissue cell, a spleen cell, heart cell, kidney cell, a testis cell, or an intestinal tract cell.

5. The method of claim 1, wherein the GDE protein comprises one or more GDE family proteins.

6. The method of claim 1, wherein the GDE protein comprises glycerophosphodiester phosphodiesterase 2 (GDE2).

7. The method according to claim 4, wherein differentiation is up-regulatable by GDE protein over-expression.

8. The method of claim 4, wherein differentiation is down-regulatable by reducing the functional level of GDE protein level.

9. The method according to claim 3, wherein the condition is one or more cancer, infertility, pulmonary disease, tissue engineering, nerve damage, gastrointestinal disease, pain, trauma, migraine, neurological disorders, cardiovascular conditions, diabetes, cancer, drug addiction, analgesic side effect, analgesic tolerance, diabetes, infertility, neurodegenerative disorders or a behavioral disorder.

10. The method according to claim 1, wherein the modulation is up-regulation of a GDE protein level and the up-regulation comprises introducing a nucleic acid molecule encoding a GDE protein or functional equivalent, derivative or homologue thereof or the GDE protein expression product or functional derivative, homologue, analogue, equivalent or mimetic thereof to the cell.

11. The method according to claim 1, wherein the modulation comprises contacting the cell with a compound that modulates transcriptional and/or translational regulation of a GDE gene.

12-15. (canceled)

16. A method of converting a stem cell into a ventral neuron which comprises introducing into the stem cell a nucleic acid which expresses homeodomain transcription factor Nkx6.1 protein in the stem cell so as to thereby convert the stem cell into the ventral neuron.

17. A method of converting a motor neuron progenitor into a post-mitotic neuron comprising introducing a nucleic acid expressing a GDE protein into the motor neuron progenitor to thereby convert the stem cell into the post-mitotic neuron.

18. The method of claim 17, wherein the nucleic acid incorporates into the chromosomal DNA of the cell.

19-20. (canceled)

21. A pharmaceutical composition comprising a pharmaceutically effective amount of a GDE modulator effective to treat, prevent, ameliorate, reduce or alleviate a GDE related disorder or symptoms thereof and a pharmaceutically acceptable excipient.

22-23. (canceled)

24. A vector encoding one or more GDE proteins or fragments or variants thereof, or an isolated cell that recombinantly expresses one or more peptides identified by SEQ ID NO. 1, or fragments or variants thereof.

25. (canceled)

26. A method to treat, prevent, ameliorate, reduce or alleviate a GDE related disorder or symptoms thereof, comprising: administering to a subject in need thereof a composition comprising a pharmaceutically effective amount of a GDE modulator.

27-36. (canceled)

37. A method for identifying lead compounds for a pharmacological agent useful in the treatment of a GDE related disorder comprising:
contacting a cell expressing a GDE protein with a test compound, and
measuring GDE expression or differentiation or modulation of GDPD activity.

38-41. (canceled)

42. A transgenic non-human animal comprising an over-expressed GDE protein or a fragment or variant thereof.

43. (canceled)

44. A method for screening a therapeutic agent to treat, prevent, ameliorate, reduce or alleviate a GDE related disorder or symptoms thereof, comprising:
administering a test agent to a mouse having an over-expressed GDE protein, and
measuring modulation of differentiation.

45-46. (canceled)
